SEQUENCE LISTING (4 sequences)

SEQ ID NO: 1 - DNA, 21 bp, Artificial Sequence
  Synthetic
cgttacctgtgtgtggtgata

SEQ ID NO: 2 - DNA, 23 bp, Artificial Sequence
  Synthetic
ctctggtcctggtatgaagaatg

SEQ ID NO: 3 - DNA, 22 bp, Artificial Sequence
  Synthetic
cccttcattgacctcaactaca

SEQ ID NO: 4 - DNA, 20 bp, Artificial Sequence
  Synthetic
atgacaagcttcccgttctc

(19) United States
(12) Patent Application Publication
(10) Pub. No.: US 2022/0372441 A1
(43) Pub. Date: Nov. 24, 2022

(54) MICRORNAS ENRICHED IN MEGAKARYOCYTIC EXTRACELLULAR VESICLES AND USES THEREOF

(71) Applicants: Eleftherios Papoutsakis, Newark, DE (US); Chen-Yuan Kao, Newark, DE (US); Jinlin Jiang, Rockville, MD (US)

(72) Inventors: Eleftherios Papoutsakis, Newark, DE (US); Chen-Yuan Kao, Newark, DE (US); Jinlin Jiang, Rockville, MD (US)

(73) Assignee: University of Delaware, Newark, DE (US)

(21) Appl. No.: 17/770,433

(22) PCT Filed: Oct. 21, 2020
(86) PCT No.: PCT/US2020/056593

§ 371 Date: Apr. 20, 2022

Related U.S. Application Data
(60) Provisional application No. 62/923,841, filed on Oct. 21, 2019.

(51) Int. Cl. C12N 5/078; C12N 15/113

(52) U.S. Cl. CPC C12N 5/0644; C12N 15/113; C12N 2506/11; C12N 2310/141; C12N 2501/65; C12N 2527/00

(57) ABSTRACT

The present invention relates to a method for inducing megakaryocytic differentiation of hematopoietic stem/progenitor cells (HSPCs). The method comprises transferring into the HSPCs an effective amount of small RNAs. The HSPCs may differentiate into megakaryocytes in the absence of thrombopoietin (TPO) and/or without using megakaryocytic microparticles (MkMPs). The small RNAs may be micro RNAs (miRs) selected from the group consisting of miR-486, miR-22, miR-191, miR-181, miR-378, miR-26, let-7, miR-92, miR-126, miR-92, miR-21, miR-146, miR-181, and combinations thereof. For example, the small RNAs are miR-486 and miR-22. The small RNAs may be synthetic or isolated from cells. Also provided is a method for enhancing megakaryocytic differentiation of HSPCs cultured with megakaryocytic microparticles MkMPs in the presence of an effective amount of one or more exogenous small RNAs (e.g., miR-486).

### CROSS-REFERENCE TO RELATED APPLICATION

This application claims priority to U.S. Provisional Application No. 62/923,841, filed Oct. 21, 2019, and the contents of which are incorporated herein by reference in their entireties for all purposes.

### REFERENCE TO U.S. GOVERNMENT SUPPORT

This invention was made with government support under Grant No. 1804741 awarded by the U.S. National Science Foundation. The United States has certain rights in the invention.

### FIELD OF THE INVENTION

The invention relates generally to small RNAs enriched in megakaryocytic extracellular vesicles and uses thereof for inducing megakaryocytic differentiation of hematopoietic stem/progenitor cells (HSPCs).

### BACKGROUND OF THE INVENTION

Cell-derived microparticles (MPs) are sub-micron size (0.1-1.0 μm) extracellular vesicles (EVs) that play an important role in cell-to-cell communication by carrying and transferring native cargo, including proteins, lipids, and RNAs to target cells. Cargo delivery triggers the development of complex phenotypes through mechanisms involving signaling and, broadly, regulation of gene expression.

Megakaryocytic microparticles (MkMPs) can induce Megakaryocytic (Mk) differentiation of mobilized peripheral blood CD34⁺ Hematopoietic Stem and Progenitor cells (HSPCs) in the absence of exogenous Thrombopoietin (TPO), yet similar in potency and effect as TPO. Although MkMPs are highly enriched in small RNAs, this observed effect of MkMP in inducing megakaryocytic differentiation of HSPCs cannot be yet scientifically assigned to any specific molecule. The scientific evidence would broadly assign this effect to all or many undefined cargo molecules (RNAs, proteins, lipids) of MkMPs and not to a specific, identifiable microRNA, protein or lipid. RNase treatment, differentially depleting the small RNA pool, attenuated the ability of MkMPs to trigger Mk differentiation of HSPCs, thus suggesting that small RNAs, possibly microRNAs (miRs), can mimic TPO signaling in HSPCs, a hitherto unknown possibility and mechanism. Still, given the thousands of miRs that are likely contained in MkMPs, the observed effect of MkMP in inducing megakaryocytic differentiation of HSPCs cannot be yet scientifically assigned to any specific miR or other RNA, small of large. TPO-induced signaling starts with the binding of TPO to its receptor c-Mpl, which activates Janus-family kinases (Jaks). Downstream signaling pathways include signal transducers and activators of transcription (STAT), mitogen-activated protein kinases (MAPKs), and notably phosphoinositide 3-kinase (PI3K)/Akt/mammalian target of rapamycin (mTOR). Within the MAPK family, the MEK-ERK1/2 (MAPK kinase-extracellular signal-related kinases 1 and 2) signaling has been shown to play an important role in TPO-induced Mk development, while p38-MAPK was shown to be involved in TPO-mediated hematopoietic stem cell (HSC) expansion and erythropoiesis. Although TPO have been shown to activate c-Jun amino-terminal kinases (JNKs) signaling, there is no known role for JNK in TPO-induced Mk development.

miRs are small non-coding RNAs regulating gene expression at the post-transcriptional level by targeting specific mRNAs leading to mRNA degradation or translational inhibition. EV-mediated transfer of miRs between cells has been studied in various cell types. Single-molecule real-time (SMRT) RNA sequencing (RNAseq) was used to identify specific RNAs involved in EV-triggered phenotypes of target cells. In these and other studies, a single EV miR was identified as responsible for the biological phenotype. There is increasing evidence however that two or more miRs are involved in co-regulating the same biological program or process in cancer and normophysiology. Combinations of miRs have been also used synthetically to regulate biological processes. However, cooperation between miRs from an EV has been rarely examined. Recently, a group of EV-associated miRs have been reported to likely mediate pro-inflammatory cytokine production in a murine sepsis model, but mechanistic understanding was not pursued.

There remains a need for a simple and effective method to induce or enhance megakaryocytic differentiation in hematopoietic stem/progenitor cells (HSPCs).

### SUMMARY OF THE INVENTION

The present invention relates to small RNAs, for example, microRNAs (miRs), enriched in megakaryocytic extracellular vesicles and uses thereof. The inventors have surprisingly discovered that such small RNAs may be used for inducing megakaryocytic differentiation of hematopoietic stem/progenitor cells (HSPCs), for example, in the absence of exogenous Thrombopoietin (TPO) and/or without using megakaryocytic microparticles (MkMPs), or enhancing megakaryocytic differentiation of HSPCs cultured with MkMPs.

A method for inducing megakaryocytic differentiation of hematopoietic stem/progenitor cells (HSPCs) without megakaryocytic microparticles (MkMPs) is provided. The method comprises transferring into the HSPCs an effective amount of one or more small RNAs such that the HSPCs differentiate into megakaryocytes. The method may not comprise using MkMPs carrying the one or more small RNAs. The HSPCs may be CD34⁺. In one embodiment, the HSPCs may differentiate into megakaryocytes in vitro in the absence of thrombopoietin (TPO). In another embodiment, the HSPCs may be in a subject and differentiate into megakaryocytes in the subject.

The one or more small RNAs may comprise one or more microRNAs (miRs). The miRs may be selected from the group consisting of miR-486, miR-22, miR-191, miR-181, miR-378, miR-26, let-7, miR-92, miR-126, miR-92, miR-21, miR-146, miR-181, and combinations thereof. The one or more small RNAs may comprise miR-486 and/or miR-22. The one or more small RNAs may further comprise hsa_piR-001312, hsa_piR-000765, hsa_piR-020326, hsa_piR-016658, hsa_piR-017724 or a combination thereof. The one or more small RNAs may further comprise SNORD29, SNORD68, SNORD104, SNORD42A, SNORD26, SNORD99, SNORD44, SNORD50A, SNORD43, SNORD2 or a combination thereof. The one or more small RNAs may be synthetic. The one or more small RNAs may be isolated from cells.

The method may further comprise transferring the one or more small RNAs into the HSPCs via cellular particles. The cellular particles may be generated by a preparation method. The preparation method may comprise culturing cells in a culture medium. The cells may be selected from the group consisting of megakaryocytes, immature megakaryocyte cells and a combination thereof. The preparation method may further comprise exposing the cultured cells to a mechanical stress to generate cellular particles, isolating the cellular particles from the cultured cells, and loading the isolated cellular particles with the one or more small RNAs.

The method may further comprise transferring the one or more small RNAs into the HSPCs by transfection, electroporation, lipofection or nucleofection.

The method may further comprise administering to the HSPCs a regulator of PI3K signaling pathway. The regulator of the PI3K signaling pathway may be a positive regulator such that the megakaryocytic differentiation of the HSPCs is enhanced.

The method may further comprise administering to the HSPCs a regulator of Akt signaling pathway. The regulator may be a positive regulator such that the megakaryocytic differentiation of the HSPCs is enhanced.

A method for enhancing megakaryocytic differentiation of hematopoietic stem/progenitor cells (HSPCs) is provided. The method comprises culturing HSPCs with MkMPs in the presence of an effective amount of one or more exogenous small RNAs. More of the HSPCs differentiate into megakaryocytes than those cultured with the MkMPs in the absence of the one or more exogenous small RNAs. The one or more exogenous small RNAs may comprise miR-486. The method may further comprise loading the MkMPs with the one or more exogenous small RNAs. The method may further comprise transferring the one or more exogenous small RNAs into the HSPCs by transfection, electroporation, lipofection or nucleofection.

### BRIEF DESCRIPTION OF THE DRAWINGS

FIG. 1 is a Venn diagram showing the number of microRNAs (miRs) detected in (CPM ≥1) and shared among megakaryocytes (Mks), Megakaryocytic MPs (MkMPs), platelet-like particles (PLPs), and platelets (PLTs).

FIGS. 2A-D show distribution of top 10 miRs from 3-donor (A) Mks, (B) MkMPs, (C) PLPs, and 2-donor (D) PLTs.

FIG. 3 is a volcano plot showing the results from the differential expression analysis of miRs in MkMPs vs. Mk cells. 58 miRs were significantly (p <0.01) enriched (fold change ≥2) in MkMPs, while expression levels of 90 MkMP miRs were significantly lower (fold changes ≤0.5) than in Mk cells.

FIG. 4 is a pie chart showing the miR distribution in MkMPs. The top seven (7) miRs are shown in detail.

FIGS. 5A-C show characteristics of 200,000 CD34⁺ HSPCs transfected with 2 or 8 μM miR-486-5p or miR-22-3p mimics. (A) Cells were harvested for flow cytometric analysis on CD41 expression at day 10. (B) An example of using FITC IgG antibody control to determine the CD41⁺ population in histograms. (C) Cells transfected with 8 μM miR-486-5p were also harvested at day 10 for flow cytometric analysis with a quadrant gate for CD41 and CD61 expression. Error bars in (A) represent standard error of mean of 2 biological replicates. *p<0.05.

FIGS. 6A-C show the effect of single miRs on Mk differentiation. CD34⁺ HSPCs were transfected with 8 μM miR mimics (N=2), miR negative control (miR-NC), or without miRs (No miR), and cells were cultured in minimal medium (IMDM supplemented with 10% BIT and 50 ng/ml SCF) but without Tpo. Cells cultured in Tpo-supplemented medium (100 ng/ml Tpo) served as positive control (TPO). Cells were harvested for flow cytometric analysis for (A) CD41 expression at day 13. (B) Total cell counts and (C) total Mk-cell counts at day 10. Error bars represent the standard error of mean from 2 biological replicates. *p<0.05.

FIGS. 7A-G show the effect of single or multiple miRs on Mk differentiation. CD34⁺ HSPCs were transfected with miR mimics (N=8), miR negative control (miR-NC, N=8), or without miRs (No miR, N=8), and cells were cultured in minimal medium (IMDM supplemented with 10% BIT and 50 ng/ml SCF) without TPO. Cells cultured in TPO-supplemented medium (100 ng/ml TPO, N=6) or cells co-cultured with MkMPs (N=3) served as positive controls (TPO, MkMP). Cells were harvested for flow cytometric analysis for (A, B) CD41 expression at days 7, 10 and 13, and (C) CD42b expression at day 10. (D) Representative flow cytometric dot plots for CD41 and CD42b expression at day 7, 10 and 13 with quadrant gates. (E) Mk (CD41⁺) cell numbers at day 13, or (F) total cell numbers at day 10 were measured by flow cytometry, respectively. (G) At day 16, cells were harvested for ploidy analysis by flow cytometric analysis. Error bars in (A, B, C, E, F, G) represent the standard error of mean from 3-8 biological replicates. Statistical comparison analysis was performed between each experimental group against the two negative controls (No miR or miR-NC) unless otherwise shown on panel C. *p<0.05, **p<0.01, ***p<0.001. Electroporation of CD34⁺ HSPCs damages and alters the properties of CD34⁺-cell membrane thus resulting in attenuated MkMP-induced Mk differentiation of CD34⁺ HSPCs (data not shown).

FIGS. 8A-C show the effect of single miRs or miR pairs on cell expansion. CD34⁺ HSPCs were transfected with miR mimics (N=8), miR negative control (miR-NC, N=8), or without miRs (No miR, N=8), and cells were cultured in minimal medium (IMDM supplemented with 10% BIT and 50 ng/ml SCF) without TPO. Cells cultured in TPO-supplemented medium (100 ng/ml TPO, N=6) or cells co-cultured with MkMPs (N=3) served as positive control (TPO, MkMP). The percent of CD41⁺ CD42⁺ at day 10 (A) and day 13 (B), and total cell numbers (C) at day 7 were measured by flow cytometry. miR-22-3p significantly promotes cell proliferation. Error bars represent the standard error of mean from 3-8 biological replicates. * represent the comparison to negative controls (No miRNA or miR-NC) unless otherwise indicated on panels A and B. *p<0.05, **p<0.01.

FIG. 9 shows that miR-486-5p and miR-22-3p enable Mk differentiation and maturation. CD34+ cells were transfected with miRNA mimics, non-targeting miRNA (miR-NC), or without miRNAs (No miR), and were cultured in minimal medium (IMDM supplemented with 10% BIT and 50 ng/ml SCF) without TPO. Cells cultured in TPO-supplemented medium (100 ng/ml TPO) served as positive control (TPO). Cells were harvested at day 13 and stained for 5-HT (cyan), vWF (red), TUBB1 (green), and DAPI (blue), and images were acquired by confocal microscopy. Scale bar: 10 μm. White arrows represent the pre-demarcation membrane system (DMS) structure. White open arrows represent the proplatelet-like structure.

FIGS. 10A-B show that loading of miR-486-5p inhibitor into MkMPs reduces the native effect of MkMPs in inducing Mk differentiation of CD34⁺ HSPCs. (A) CD34⁺ HSPCs were co-cultured with MkMPs, 8 μM-miR-486-5p inhibitor-loaded MkMPs, 8 μM-miR-22-3p inhibitor-loaded MkMPs, or vehicle control. Cells were harvested for flow cytometric analysis on CD41 expression at day 4, 7, and 10. (B) MkMPs, or 16 ρM-miR-486-5p inhibitor-loaded MkMPs were co-cultured with CD34⁺ HSPCs. Total cell or Mk cell (CD41⁺ cells) numbers were measured at day 10.

FIGS. 11A-F show the impact of signaling inhibitors on MkMP-induced Mk differentiation. CD34⁺ HSPCs were pretreated with a signaling inhibitor, or solution without an inhibitor (MkMP control), and were co-cultured with MkMPs or vehicle control. Cells were harvested for flow cytometric analysis for (A, B) CD41 expression at day 7 and day 12, (C) CD42b expression at day 12, or (D) CD34 expression at days 4, 7, and 12. (E) Representative flow cytometric dot plots for CD34 and CD41 expression at day 4, 7 and 12 with quadrant gates. (F) Total cell numbers were measured at day 7. Error bars in (A-C) represent the standard error of mean of 6-8 biological replicates, while error bars in (D-F) represent the standard error of mean of 3 biological replicates *p<0.05, **p<0.01 (compared to MkMP control)

FIGS. 12A-E show that MkMPs activate PI3K/Akt/mTOR signaling in CD34⁺ HSPCs mediated by JNK signaling. (A, B) CD34⁺ HSPCs were co-cultured with MkMPs or vehicle control. Cells were harvested after 24 hours of co-culture, and the level of targets including mTOR, p-mTOR, and Akt were examined by immunoblotting. (A) Representative immunoblot images out of 4 replicates. (B) Semi-quantification of p-mTOR expression from immunoblot images (n=4). (C-E) CD34⁺ HSPCs were pretreated with a signaling inhibitor (JNK, Akt, or mTOR inhibitor), or inhibitor vehicle (without an inhibitor), and were co-cultured with MkMPs or without (vehicle control). Cells were harvested after 24 hours of co-culture, and the level of p-mTOR, Akt, and p-Akt were examined and quantified by flow cytometric analysis (n=3-5). Representative histograms of each target are shown on the right. Error bars in (B) represent the standard error of mean of 2-4 biological replicates, while error bars in (C-E) represent the standard error of mean of 3-5 biological replicates. Statistical comparison analysis was performed between each experimental group against MkMP control (without inhibitor). *p<0.05, **p<0.01, ***p<0.001.

FIGS. 13A-D show that MkMPs target PTEN and is mediated by JNK signaling. (A-C) CD34⁺ HSPCs pretreated with JNK inhibitor or solution without an inhibitor were co-cultured with MkMPs or vehicle control for 24 hours. (A) PTEN protein expression was quantified by flow cytometric analysis, while (B) PTEN mRNA levels were quantified by qPCR analysis. (C) PTEN expression was examined by immunoblotting (D) Schematic diagram of signaling pathway triggered by MkMPs in CD34⁺ HSPCs, based on the results from (A-C) and (A-E) from FIG. 12. Error bars in (A, B) represent the standard error of mean of 3-5 biological replicates. Statistical comparison analysis was performed between each experimental group against MkMP control (without inhibitor). *p<0.05, **p<0.01, ***p<0.001.

FIG. 14 is a schematic diagram of proposed model for MkMP-induced signaling in CD34⁺ HSPCs and its relationship to miRs in MkMPs. The model is based on the following pieces of evidence. Our data showed that co-culture of CD34⁺ HSPCs with MkMPs activates PI3K/Akt/mTOR and JNK signaling pathway toward cell expansion and megakaryocytic differentiation. Specifically, JNK and mTOR signaling are involved in late Mk differentiation (CD42b expression). A crosstalk between JNK and PI3K/Akt/mTOR was identified via the negative regulator of PI3K/Akt/mTOR signaling, PTEN. PI3K and PTEN were known major targets of miR-486-5p.

### DETAILED DESCRIPTION OF THE INVENTION

The present invention provides a novel method for inducing megakaryocytic differentiation of hematopoietic stem/progenitor cells (HSPCs). The inventors have surprisingly discovered that microRNAs (miRs) highly enriched in megakaryocytic microparticles (MkMPs) are differentially expressed in MkMPs, compared to miRs from megakaryocytes (Mks), and can be used for promoting thrombopoiesis, megakaryopoiesis and cell proliferation. The present invention also provides a mechanistic understanding of the biological effect of MkMPs in inducing megakaryocytic differentiation of HSPCs, which is a phenotype of potential physiological significance in stress megakaryopoiesis. This invention further provides one or multiple miRs from MkMPs, analyzed from single-molecule real-time (SMRT) RNA sequencing (RNAseq), and their uses thereof in the application of promoting thrombopoiesis and megakaryopoiesis.

The present invention discloses the miR profile (from RNA sequencing analysis) of MkMPs, and its comparison to the miR profile of platelet-like particles (PLPs), their parent Mk cells, and platelets (PLTs). The present invention also discloses that abundant miRs, specifically top 10 miRs, from MkMPs can be used for the application to promote thrombopoiesis, megakaryopoiesis and cell proliferation. The present invention further discloses that highly-enriched miRs, from MkMPs can be used for the application to promote thrombopoiesis, megakaryopoiesis and cell proliferation. These miRs are differentially expressed in MkMPs, compared to miRs from Mks. The present invention discloses synergetic effect of two or more miRs from MkMPs in inducing Mk differentiation, promoting thrombopoiesis and cell proliferation, as well as the signaling pathways associated to the miRs from MkMPs. The present invention further relates to all biological application ex vivo or in vivo of miRs from MkMPs in inducing megakaryocytic differentiation of HSPCs leading to de novo platelet biogenesis and thrombopoiesis to enhance vascular repair and ameliorate induced (such as due to chemotherapy, radiation or generally drugs) or idiopathic thrombocytopenia (low platelet count). Using one or a combination of miRs constitutes a precise therapeutic composition that targets the desirable phenotype. MkMPs and all MPs or EVs contain thousands of molecules (proteins, RNAs, lipids, small metabolites, etc.) of unknown composition and unknown impact individually on the cells and the desirable phenotype.

The term “hematopoietic stem/progenitor cells (HSPCs)” as used herein refers cells present in or isolated from blood and bone marrow that are capable of forming mature blood cells, for example, red blood cells, platelets or white blood cells. The HSPCs may be CD34⁺, CD34⁺, CD59⁺, CD90/Thy1⁺, CD38^{low/-}, c-Kit^{-/low}, and/or Lin⁻.

The term “megakaryocytes (MK)” as used herein refers to large bone marrow cells with a lobated nucleus responsible for production of blood thrombocytes (i.e., platelets). Megakaryocytes are derived from HSPCs in the bone marrow. Megakaryocytes may be CD41⁺, CD42b⁺ and/or CD61⁺.

The term “megakaryocytic differentiation of hematopoietic stem/progenitor cells (HSPCs)” as used herein refers to forming megakaryocytes by HSPCs. The megakaryocytic differentiation may be evidenced by expression of CD41, CD42b and/or CD61. The megakaryocytic differentiation may also be evidenced by the generation of Mk cells with high ploidy (polyploidization) through the endomitotic cycle.

The term “megakaryocytic microparticles (MkMPs)” used herein refers to the large extracellular vesicles (EV) released by megakaryocytes as generated, characterized and described in Jiang, Kao and Papoutsakis (J. Controlled Release, 2017; 247:1-18). The MkMPs contain native cellular content and/or native small RNAs, for example, microRNAs (miRs), but also large RNAs, proteins and lipids. The MkMPs may be isolated from megakaryocytes as described in Jiang, Kao and Papoutsakis (J. Controlled Release, 2017; 247:1-18) and Jiang, Woulfe and Papoutsakis (Blood, 2014; 124(13):2094-2103). MkMPs may express the same markers as the megakaryocytes.

The term “small RNA” used herein refers to a RNA molecule having fewer than 200 nucleotides. The small RNAs may be present in MkMPs in an amount at least 50%, 60%, 70%, 80%, 90%, 100%, 200% or 500% greater than that in MKs. The small RNA may be a non-coding RNA molecule. The small RNA may be a microRNA (miR), Piwi-interacting RNA (piRNA), small nucleolar RNA (snoRNA), non-snoRNA, or a combination thereof.

The term “subject” used herein refers to a mammal, for example a human. The subject may be a patient. The subject may have suffered from a disease or disorder associated with lack of Mk. The subject may be in needed of Mks or platelets.

The present invention provides a method for inducing megakaryocytic differentiation of hematopoietic stem/progenitor cells (HSPCs). The method comprises transferring into the HSPCs an effective amount of one or more small RNAs. As a result, the HSPCs differentiate into megakaryocytes. The HSPCs may differentiate into megakaryocytes in the absence of Thrombopoietin (TPO) and/or Thrombopoietin receptor agonists (TPO-RAs) (e.g., Romiplostim and Eltrombopag). The HSPCs may differentiate into megakaryocytes without using megakaryocytic microparticles (MkMPs).

The HSPCs may be isolated from a subject. The HSPCs may differentiate into megakaryocytes in vitro, for example, in the absence of thrombopoietin (TPO). At least 50%, 60%, 70%, 80%, 90%, 95% or 99% the HSPCs may differentiate within a predetermined time period, for example, 1, 2, 3, 4, 5, 6, 7, 8, 9, 10, 11, 12, 13, or 14 days, after the transfer.

The HSPCs may be in a subject and differentiate into megakaryocytes in the subject. The megakaryocytes may then form platelets, which may be therapeutic. The megakaryocytic differentiation may occur within a predetermined time period, for example, 1, 2, 3, 4, 5, 6, 7, 8, 9, 10, 11, 12, 13, or 14 days, after the transfer. Thrombopoietin (TPO) may not be administered to the subject.

According to the present invention, the small RNAs may comprise microRNAs (miRs). The miRs may be enriched in the MkMPs. The miRs may be selected from the group consisting of miR-486, miR-22, miR-191, miR-181, miR-378, miR-26, let-7, miR-92, miR-126, miR-92, miR-21, miR-146, miR-181, and combinations thereof. In one embodiment, the small RNAs comprise miR-486 (e.g., miR-486-5p) and/or miR-22 (e.g., miR-22-3p), which may have a synergistic effect on megakaryocytic differentiation of the HSPCs.

The small RNAs may further comprise other small RNAs. The other small RNAs may be enriched in the MkMPs. The other small RNAs may be selected from the group consisting of hsa_piR-001312, hsa_piR-000765, hsa_piR-020326, hsa_piR-016658, hsa_piR-017724 and combinations thereof. The other small RNAs may be selected from the group consisting of SNORD29, SNORD68, SNORD104, SNORD42A, SNORD26, SNORD99, SNORD44, SNORD50A, SNORD43, SNORD2, and combinations thereof. The other small RNAs may be transferred into the HSPCs with the miRs concurrently or sequentially.

The small RNAs may be synthetic. The small RNAs may be isolated from cells, for example, megakaryocytes, immature megakaryocyte cells or a combination thereof. The small RNAs may be stable. At least 50%, 60%, 70%, 80%, 90%, 95% or 99% of the small RNAs may remain intact at a temperature of, for example, 0-37° C., 1-4° C., 1-20° C., 1-25° C., 4-20° C., 4-25° C. or 4-37° C. for at least 1, 2, 3, 4, 5, 6, 7, 8, 9, 10, 11, 12, 13, or 14 days. The small RNAs may retain at least 50%, 60%, 70%, 80%, 90%, 95% or 99% of a biological activity, for example, induction of megakaryocytic differentiation of hematopoietic stem/progenitor cells (HSPCs), at a temperature of, for example, 0-37° C., 1-4° C., 1-20° C., 1-25° C., 4-20° C., 4-25° C. or 4-37° C. for at least 1, 2, 3, 4, 5, 6, 7, 8, 9, 10, 11, 12, 13, or 14 days.

According to the present invention, the small RNAs may be transferred into the HSPCs via cellular particles. The cellular particles may be generated by culturing cells in a culture medium, exposing the cultured cells to a mechanical stress to generate cellular particles, isolating the cellular particles from the cultured cells, and loading the isolated cellular particles with the small RNAs. The cells may be selected from the group consisting of megakaryocytes, immature megakaryocyte cells and a combination thereof. The mechanical stress may be shear stress or other hydrodynamic stress.

The cellular particles may contain native cellular contents from the cultured cells. The native cellular contents may comprise proteins, RNAs, lipids, and/or small molecules of metabolic cellular intermediates from the cultured cells.

The cellular particles carrying the small RNAs may be further loaded with exogenous cellular contents. The exogenous cellular contents may comprise membranes, proteins, RNAs, lipids, and/or small molecules of metabolic cellular intermediates. The membrane may include surface proteins or receptors that recognize and target HSPCs and more differentiated blood cells deriving from HSPCs.

The small RNAs may be transferred into the HSPCs by transfection, electroporation, lipofection or nucleofection, concurrently or sequentially.

According to the present invention, the megakaryocytic differentiation of the HSPCs may be regulated by modulating one or more signaling pathways targeted by the small RNAs. The signaling pathways may be comprise PI3K signaling pathway, Akt signaling pathway or a combination thereof. The method may further comprise administering to the HSPCs an Akt signaling pathway may be a positive regulator so that the megakaryocytic differentiation of the HSPCs may be enhanced.

A method for enhancing megakaryocytic differentiation of hematopoietic stem/progenitor cells (HSPCs) is also provided. The enhancement method comprises culturing HSPCs with MkMPs in the presence of an effective amount of one or more exogenous small RNAs. More of the HSPCs differentiate into megakaryocytes than those cultured with the MkMPs in the absence of the one or more exogenous small RNAs. For example, at least 10%, 20%, 30%, 40%, 50%, 60%, 70%, 80%, 90%, 100%, 150% or 200% more of the HSPCs differentiate into megakaryocytes when cultured with the MkMPs in the presence of the one or more exogenous small RNA than those cultured with the MkMPs in the absence of the one or more exogenous small RNAs.

The exogenous small RNAs may be synthetic or isolated from cells, for example, megakaryocytes, immature megakaryocyte cells or a combination thereof. The small RNAs may comprise microRNAs (miRs). The miRs may be selected from the group consisting of miR-486, miR-22, miR-191, miR-181, miR-378, miR-26, let-7, miR-92, miR-126, miR-92, miR-21, miR-146, miR-181, and combinations thereof. In one embodiment, the small RNAs comprise miR-486 (e.g., miR-486-5p) and/or miR-22 (e.g., miR-22-3p), which may have a synergistic enhancement effect on megakaryocytic differentiation of the HSPCs. The small RNAs may further comprise other small RNAs enriched in the MkMPs. The other small RNAs may be selected from the group consisting of hsa_piR-001312, hsa_piR-000765, hsa_piR-020326, hsa_piR-016658, hsa_piR-017724 and combinations thereof. The other small RNAs may be selected from the group consisting of SNORD29, SNORD68, SNORD104, SNORD42A, SNORD26, SNORD99, SNORD44, SNORD50A, SNORD43, SNORD2, and combinations thereof. The other small RNAs may be transferred into the HSPCs with the miRs concurrently or sequentially.

The enhancement method may further comprise loading the MkMPs or with the one or more exogenous small RNAs.

The enhancement method may further comprise transferring the one or more exogenous small RNAs into the HSPCs by transfection, electroporation, lipofection or nucleofection. The HSPCs may be transfected with the one or more small RNAs before or during the culturing step.

While the present disclosure may be susceptible to embodiments in different forms, and herein various embodiments will be described in detail with the understanding that the present description is to be considered an exemplification of the principles of the disclosure and is not intended to be exhaustive or to limit the disclosure to the details of construction and the arrangements of the components set forth in the description or illustrated in the drawings.

### Example 1. miR-486-5p and miR-22-3p Enable Megakaryocytic Differentiation of Hematopoietic Stem and Progenitor Cells without Thrombopoiet

Materials and Methods

Materials: Recombinant human interleukin 3 (IL-3), IL-6, IL-9, IL-11, stem cell factor (SCF), and thrombopoietin (TPO) were purchased from PeproTech Inc. BIT 9500 was purchased from Stemcell Tech. Anti-CD61 magnetic microbeads and MACS cell-separation tools were purchased from Miltenyi. Fluorescein isothiocyanate (FITC)-conjugated anti-CD41, Phycoerythrin (PE)-conjugated anti-CD42b, allophycocyanin (APC)-conjugated anti-CD34, and IgG antibodies were purchased from BD bioscience. Signaling inhibitors, miRNA mimics, and miR-negative control were purchased from Sigma-Aldrich.

Generation of Megakaryocytic MPs (MkMPs) from cultured Megakaryocytes (Mks) starting with CD34⁺ HSPCs: CD34⁺-derived Mks were cultured as described starting with frozen G-CSF-mobilized human peripheral blood CD34⁺ cells (Fred Hutchinson Cancer Research Center). Briefly, Cells were thawed and cultured in Iscove modified Dulbecco medium (IMDM, Gibco) supplemented with 20% BIT 9500 (Stemcell Tech.), 100 ng/mL TPO, 100 ng/mL stem cell factor (SCF), 2.5 ng/mL interleukin-3 (IL-3), 10 ng/mL IL-6 & IL-11 and human LDL under 5% O₂ for 5 days. IL-3 was increased to 10 ng/mL and IL-6 was substituted with IL-9 at day 5. Cells were cultured under 20% O₂ from day 5 to 7. At day 7, in order to achieve pure megakaryocyte culture, CD61⁺ cells were enriched by using MACS separation with anti-CD61 magnetic microbeads (Miltenyi). Enriched cells were then cultured in IMDM supplemented with 20% BIT 9500, 100 ng/mL TPO, 100 ng/mL SCF, and human LDL under 20% O₂ for another 5 days. MkMPs were isolated from the culture medium of the day 12 Mk culture as described by Jiang et al. (J Control Release. 2017; 247:1-18).

Human platelets: Blood for isolation of human platelets (PLTs) was collected by venipuncture from adult healthy human volunteers after providing written informed consent as approved by the Institutional Review Board at the University of Delaware (IRB protocol #622751). Briefly, 50 mL of blood was collected into a syringe with ACD buffer (trisodium citrate, 65 mM; citric acid, 70 mM; dextrose, 100 mM; pH 4.4) at a volume ratio of 1:6 (ACD:blood). Following that, blood was centrifuged at 250×g for 10 min and the platelet-rich plasma was isolated from the supernatant. PLTs were then pelleted at 750×g for 10 min, followed by 1 wash with HEN buffer (10 mM HEPES, pH 6.5, 1 mM EDTA, 150 mM NaCl) containing 0.05 U/ml apyrase. After that, PLTs were resuspended in HEPES-Tyrode's buffer (137 mM NaCl, 20 mM HEPES, 5.6 mM glucose, 1 g/l BSA, 1 mM MgCl2, 2.7 mM KCl, 3.3 mM NaH2PO4).

RNA extraction and library preparation for RNAseq analysis: 11 small RNA libraries were prepared as described by Lu et al. (Methods. 2007; 43(2):110-117). They include 3 biological samples of Mks, MkMPs, and PLPs, and 2 biological samples of human PLTs. Total RNA was isolated using the miRNeasy micro kit (Qiagen). RNA concentration was measured by NanoDrop (Thermo Scientific, ND1000) and size distribution of total RNA was analyzed using an ABI Prism 3130XL Genetic Analyzer at the University of Delaware (UD) Sequencing & Genotyping Center at the Delaware Biotechnology Institute. Small-size RNA (18-40 nt and 40-150 nt in size) was purified by 15% polyacrylamide/urea gels and eluted from gels for library construction using Illumina TruSeq Small RNA Sample Prep kit according to the manufacturer's protocol. Briefly, RNA was sequentially ligated with 3′ and 5′ adaptors, reverse transcribed to cDNA using SuperScript III reverse transcriptase (Invitrogen) and cDNA libraries were amplified by PCR Following that, a 6% polyacrylamide gel was used to purify cDNAs with size ranges of 140-160 base pairs (bp) and 160-275 bp derived from 18-40 nt and 40-150 nt input RNA, respectively. The 11 libraries described above were pooled together. 20 μL of pooled libraries at a final concentration of 10 nM was sequenced at UD's Sequencing & Genotyping Center at Delaware Biotechnology Institute using 51 cycles on the Illumina HiSeq2500 DNA sequence analyzer.

RNAseq data analysis: Sequencing data analysis was provided by Dr. Shawn Polson and Jaysheel Bhaysar (Center for Bioinformatics & Computational Biology, UD). For small RNA (18-40 nt) sequencing data, a custom bioinformatics pipeline was used to end-trim raw reads to achieve an average quality score (Q) larger than 30, and to partition the data into miR and piRNA size fractions. Similarly, small RNA (40-150 nt) sequencing data were processed and mapped to small nucleolar RNA (snoRNA) and non-snoRNA. Only reads for which the flanking-adapter sequence was detected at the 3′ end were retained for analysis as they represent full-length sequencing of the molecule. Trimmed and filtered reads were then clustered if containing identical sequence and each cluster was aligned against human miR sequences downloaded from the miRBase (Release 21). miR reads were normalized by the number of counted reads per 1,000,000 total reads (Count per million, CPM). Differential expression analysis was performed using the edgeR Bioconductor Package. The p-value was corrected by False Discovery Rate (FDR). Corrected p value <0.01 was used to define differentially expressed miR in MkMPs.

Transfection of CD34⁺ HSPCs with miR mimics: 200,000 CD34⁺ cells were freshly thawed and cultured in IMDM supplemented with 20% BIT 9500, and 100 ng/mL SCF. After 3 hours, cells were transfected with 8 μM of miR mimics, non-targeting miR (miR-NC), or without miR (No miR) using the Amaxa Nucleofector II with program U-08. After transfection, cells were cultured in IMDM supplemented with 10% BIT 9500, 50 ng/mL SCF, and 1 ng/mL IL-3, without TPO. Cells cultured in TPO-supplemented medium (100 ng/ml TPO), or co-cultured with MkMPs served as positive controls (TPO, MkMP). The medium was replaced one day after transfection. At days 7, 10 and 13, cells were harvested for flow-cytometric analysis of CD41, CD42b expression, and Mk (CD41⁺-cell) and total cell measurements. At day 13, cells were harvested for serotonin (5-HT), von willebrand factor (vWF), beta 1 tubulin (TUBB1), and DAPI staining, as described⁷. The images were taken by ZEISS LSM 880 multiphoton confocal microscope. At day 16, cells were harvested for ploidy analysis by flow-cytometric analysis as described by Lindsey and Papoutsakis (Br J Haematol. 2011; 152(4):469-484).

miR-inhibitor experiments: Loading of miR inhibitors into MkMPs was performed as described by Kao and Papoutsakis (Sci Adv. 2018; 4:eaau6762). Briefly, MkMPs were loaded with 8 μM of miR-486-5p inhibitor, or miR-22-3p inhibitor by electroporation. 600,000 CD34⁺ HSPC cells were freshly thawed, followed by the co-culture with MkMPs or miR-inhibitor-loaded MkMPs (30 MPs per cell), or vehicle control in IMDM supplemented with 10% BIT 9500 and 50 ng/mL SCF. Cells were harvested for flow cytometric analysis of CD41 expression at day 4, 7, and 10. Total cell and Mk cell numbers were measured at day 10 of co-culture.

Signaling-inhibitor experiments: 60,000 CD34⁺ HSPCs were pretreated with signaling inhibitors for 30 min, followed by co-culture with MkMPs (30 MPs per cell) in IMDM supplemented with 10% BIT 9500 and 50 ng/mL SCF. Inhibitors were replenished at days 3 and 7. At day 4, 7 and 12, cells were harvested for flow cytometric analysis of CD41, CD42b, and CD34 expression. Total-cell and Mk counts were measured at day 7. Inhibitor concentrations and treatment times were based on published studies.

Immunoblotting: 200,000 CD34⁺ cells were first pretreated with JNK inhibitor (or DMSO as a control) for 30 min, and co-cultured with MkMPs at 30 MPs/cell, or vehicle control for 16 hours. Immunoblotting was performed described. Briefly, cells were lysed in NP-40 lysis buffer and proteins were separated by SDS-polyacrylamide gel electrophoresis using ExpressPlus 4-20% Bis-Tris polyacrylamide gels (Genscript #M42012) and the Mini-PROTEAN Tetra Vertical Electrophoresis Cell (Bio-Rad #1658004). Proteins were then transferred onto a nitrocellulose membrane (Genscript #L00224A60) via the Mini Trans-Blot® Electrophoretic Transfer Cell (Bio0Rad #1703930). Membranes were blocked using 5% milk (w/v) in TBST for 1 hour at room temperature. Primary anti-p-mTOR (Santa Cruz #sc-293133), anti-mTOR (Santa Cruz #sc-517464), anti-Akt (Santa Cruz #sc-5298), anti-PTEN (Cell Signaling, #95595), and anti-GAPDH (Santa Cruz #sc-47724) primary antibodies, and anti-rabbit Alexa Fluor 647 (Life Technologies #A21245) and anti-mouse Alexa Fluor 647 (Life Technologies #21236) secondary antibodies were used for immunoblotting. Images were captured by Typhoon FLA 9500 (GE Healthcare) and quantification of p-mTOR expression were performed by image J, normalized to the level of GAPDH.

Quantitative reverse transcription PCR (qRT-PCR): CD34⁺ HSPCs pretreated with JNK inhibitor or solution without an inhibitor were co-cultured with MkMPs, non-targeting miR-loaded MkMPs, miR-486-5p inhibitor-loaded MkMPs at 30 MPs per cell, or vehicle control. At 24 hr, cells were harvested and total RNA was isolated, reversed transcribed for qPCR assay as described by Kao and Papoutsakis (Sci Adv. 2018; 4:eaau6762). qPCR assays for PTEN and GAPDH were performed with PerfeCTa SYBR Green Supermix (QuantaBio) and the following primers: Forward (5′-CGTTACCTGTGTGTGGTGATA-3′) (SEQ ID NO: 1), Reverse (5′-CTCTGGTCCTGGTATGAAGAATG-3′) (SEQ ID NO: 2) for PTEN, and Forward (5′-CCCTTCATTGACCTCAACTACA-3′) (SEQ ID NO: 3), Reverse (5′-ATGACAAGCTTCCCGTTCTC-3′) (SEQ ID NO: 4) for GAPDH. PTEN mRNA level were quantified by normalized to GAPDH mRNA level as the reference gene.

Intracellular protein analysis by flow cytometry: 100,000 CD34⁺ cells were first pretreated with JNK, Akt, or mTOR inhibitors (or DMSO as control) for 30 min, and co-cultured with MkMPs at 30 MPs/cell, or vehicle control for 16 hours. Cells were fixed in 4% paraformaldehyde for 15 min at room temperature, followed by the permeabilization in 90% methanol for 30 min at 4° C. After washing in PBS, cells were stained with Alexa 647-conjugated anti-mTOR (#5048S), PE-conjugated anti-Akt (#8790S), or Alexa 488-conjugated anti-p-Akt (#4071S) antibodies from Cell Signaling, or Alexa 647-conjugated anti-p-mTOR (#564242) from BD bioscience, for 1 hour at room temperature, followed by flow-cytometric analysis.

Statistical analysis: Data are presented as means±standard error of mean (SEM) from at least three replicates. Paired Student's t test of all data was performed. Statistical significance is defined as *p<0.05, **p<0.01, ***p<0.001.

1. The miR content of huMkMPs is well preserved among donors.

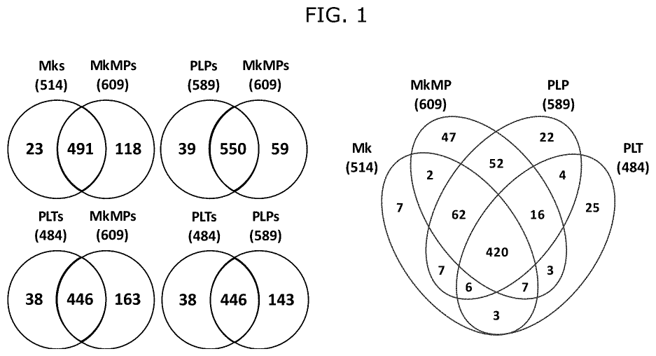

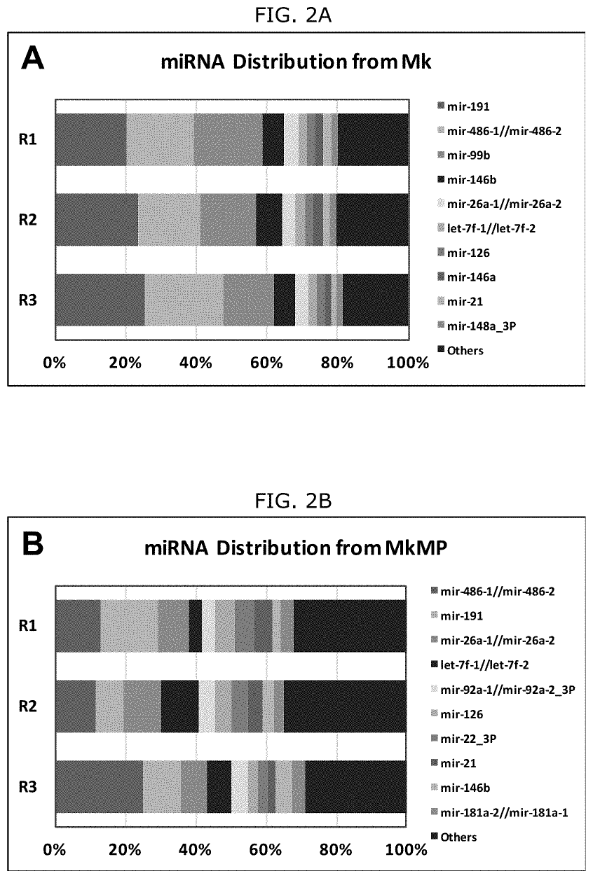

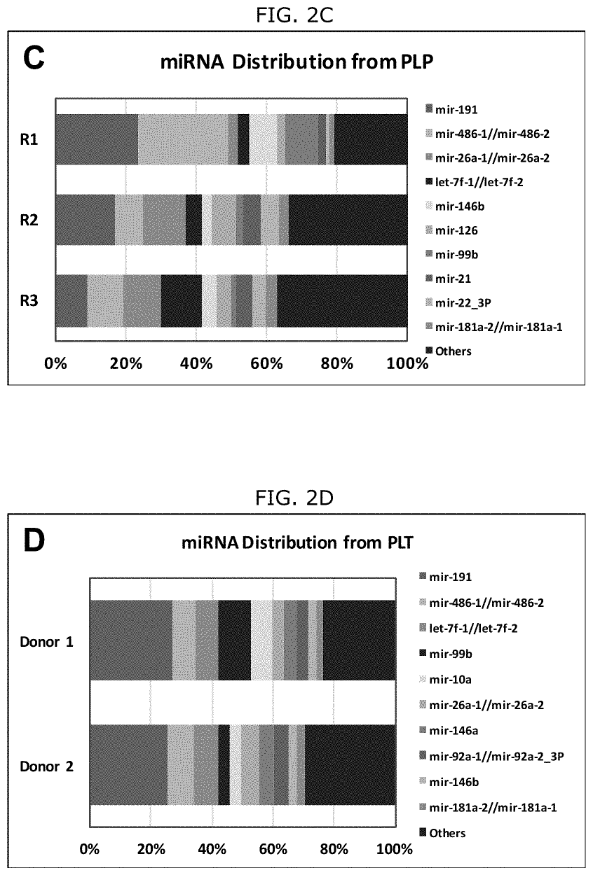

We have previously shown that MkMPs were enriched in small RNAs, which play an important role in triggering Mk differentiation of HSPCs. We hypothesized that miRs are the dominant MkMP components in inducing and promoting Mk differentiation. To characterize and carry out comparative analysis of the MkMP miR profile, total RNA was extracted from day-12 cultured Mks (starting from CD34⁺ cells of 3 donors), MkMPs and platelet-like particles (PLPs) generated from the corresponding day-12 cultured Mks. For comparison, we also extracted and analyzed RNA from human platelets (PLTs; 2 donors unrelated to the CD34⁺-cell donors). Small RNA libraries were prepared from extracted RNA for SMRT RNAseq analysis. At the average miR expression level (Count Per Million, CPM) of 1, RNAseq identified 514, 609, 589, or 484 miRs in Mks, MkMPs, PLPs, or PLTs, respectively. To identify highly expressed miRs, we used CPM 1000 as a criterion, and identified 63 miRs as highly abundant (accounting of 96.1% of total miR content). The Venn diagram (FIG. 1) shows that Mks and MkMPs share 491 miRs, while 446 miRs were shared between MkMPs with PLTs, or PLPs with PLTs. The miR MkMP profile was most distant from that of PLTs and Mks in decreasing distance order, and closest to the PLP miR profile. The miR profiles from 3 donors for Mks, MkMPs and PLPs, or two PLTs are consistent and reproducible (FIG. 2). RNAseq analysis also identified other small RNAs, such as Piwi-interacting RNA (piRNA, 18-40 nt, Table 1), small nucleolar RNA (snoRNA, 40-150 nt, Table 2) or non-snoRNA (Table 3).

2. Differential expression analysis of miRs from various sources.

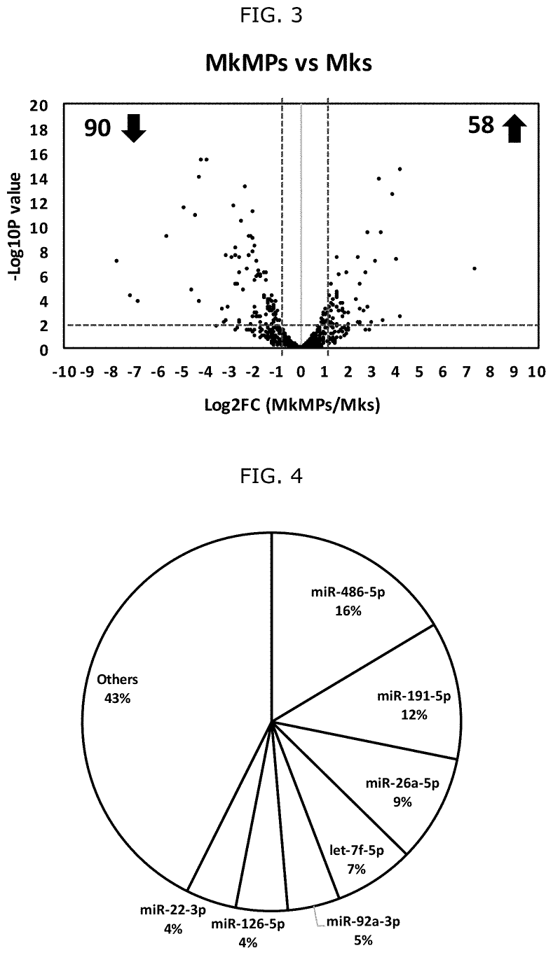

We carried out differential expression analysis of MkMP miRs against the miRs in Mk cells, PLPs and PLTs. The hypothesis was that this analysis might identify miRs that could mediate the ability of MkMPs to induce Mk differentiation of HSPCs, assuming that such miRs were also abundant in MkMPs. FIG. 3 summarizes the differential miR-expression analysis between MkMPs and Mk cells. Table 4 lists the 18 miRs which are highly enriched in MkMPs and their CPM ≥100. Only 2 miRs, mir-19b-1//mir-19b-2_3p and mir-181b-1//mir-181b-2 were within the top 50 most abundant miRs in MkMPs. Although each of the highly-enriched miRs (Table 4) only accounts for less than 0.2% of total miR content in MkMPs, these miRs are mediators of the observed phenotype.

3. Top 10 miRs making up 82% of the total miR content.

We hypothesized that highly abundant miRs in MkMPs would be more likely to achieve the observed biological phenotype. Table 5 lists the top 10 most abundant miRs in MkMPs, and for comparative purposes, also those of Mks, PLTs and PLPs. The top 20 most abundant MkMP miRs account for 82% of total miR count, while the top 7 miRs account for more than 57% of the total miR content (FIG. 4). Among the top 7 miRs in MkMP, only miR-22 is known to be involved in megakaryopoiesis by regulating the balance between erythroid and megakaryocytic differentiation in vivo. In miR-22 knock-out mice, megakaryopoiesis was enhanced after infection with lymphocytic choriomeningitis virus, while erythropoiesis was suppressed. Although there is no known role for miR-486-5p (generally known as miR-486) in megakaryopoiesis, miR-486-5p plays a role in CD34⁺-cell proliferation and erythroid differentiation.

4. Dose effect of single miR on CD34⁺ HSPCs.

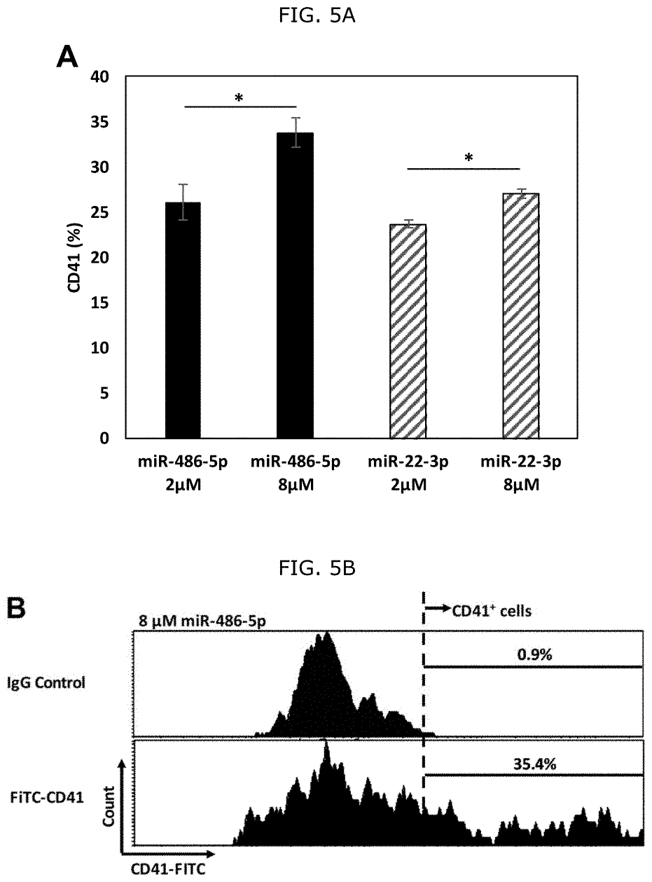

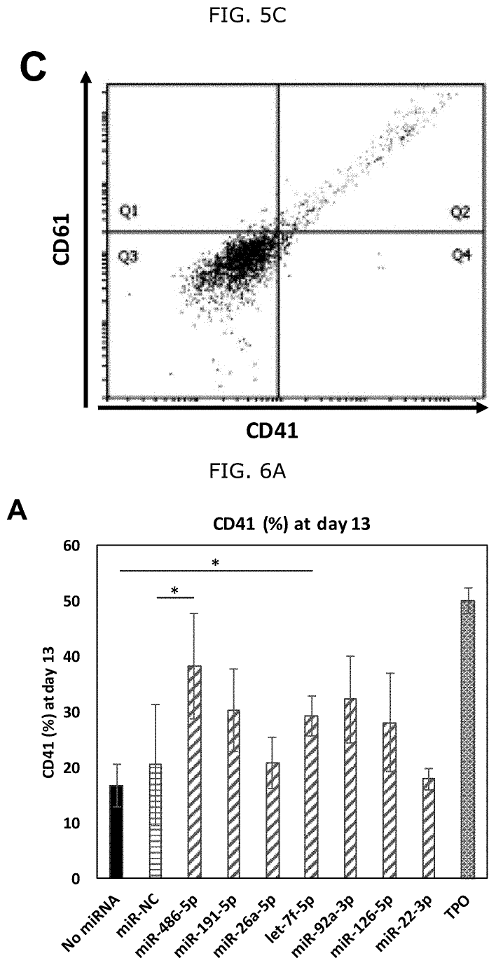

Based on previously unpublished miR data presented above, using miR mimics, we have recently demonstrated a role of miR-486-5p in Mk differentiation of CD34⁺ HSPCs. To determine if there is a dose effect of miR mimics on Mk differentiation of HSPCs, using CD41 expression as the key early Mk-differentiation marker, we carried out a pilot study with two donor CD34⁺-cell samples. CD34⁺ HSPCs were transfected with high (8 μM) or low (2 μM) concentrations of miR-486-5p or miR-22-3p (generally known as miR-22) mimics were and cultured without TPO. At day 10 of the culture, 33.8% or 27.0% of cells transfected with 8 μM miR-486-5p or 8 μM miR-22-3p were CD41⁺, while only 26.1% or 23.7% were CD41⁺ when transfected with 2 μM miR-486-5p or 2 μM miR-22-3p, respectively (FIG. 5A), thus indicating a dose effect of miRs on HSPC Mk differentiation. An example of flow cytometric analysis histograms for selecting CD41⁺ population based on proper IgG control were shown in FIG. 5B. Since CD41 and CD61 form a complex (gpIIb/IIIa), the expression level of CD41 and CD61 are virtually identical (FIG. 5C). Based on published literature, miR concentrations from 1 nM to 3.6 μM have been used to transfect HSPCs. In the following studies, since the seven MkMP miRs we examined are highly abundant in MkMPs, and due to the fact that multiple MkMPs are taken up by a single recipient CD34⁺ HSPC, we hypothesized that higher concentrations of these seven miRs are likely delivered to CD34⁺ HSPC via MkMPs. Therefore, we chose 8 μM of miR mimics for the following experiments.

5. Impact of single miR on Mk differentiation and cell proliferation of CD34⁺ HSPCs.

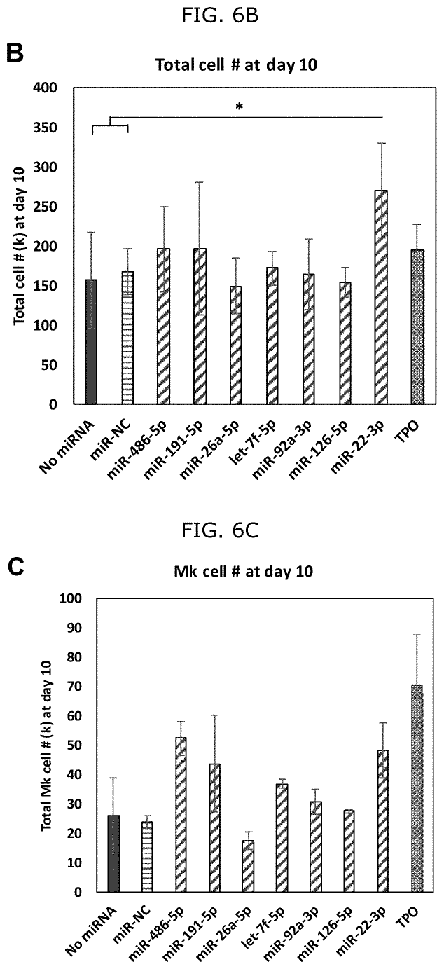

Focusing on the top 7 most abundant MkMP miRs (FIG. 4), to identify the most likely miR(s) that might impact Mk differentiation of CD34⁺ cells, we directly transfected 200,000 CD34⁺ HSPCs with 8 μM of each miR mimic separately. Transfected cells were cultured in IMDM supplemented with 10% BIT and 50 ng/ml SCF, but without TPO. Expression of CD41 and total Mk and total cell counts were examined at day 13 and 10. Negative controls were CD34⁺ cells exposed to same electroporation conditions without any miR, or with negative control miRs (miR-NC). Positive control was CD34⁺ cells exposed to same electroporation conditions cultured with 100 ng/ml TPO. Among the top 7 MkMP miRs, at day 13, compared to “miR-NC”, miR-486-5p significantly induced and promoted Mk differentiation of CD34⁺ HSPCs, achieving the highest percent (38.3%) of CD41⁺ cells, approaching that of the TPO control (FIG. 6A). The miR-22-3p mimic significantly enhanced cell expansion by up to 72% or 61% (FIG. 6B) compared to “No miR” or “miR-NC” controls, respectively. These results suggest that miR-486-5p plays a role in Mk differentiation, while miR-22-3p promotes total cell proliferation.

5. miR-486-5p in combination with miR-22-3p recapitulates in part the megakaryopoietic effect of TPO and MkMPs on CD34⁺ HSPCs, but additional enriched miRs are needed for the full effect of TPO.

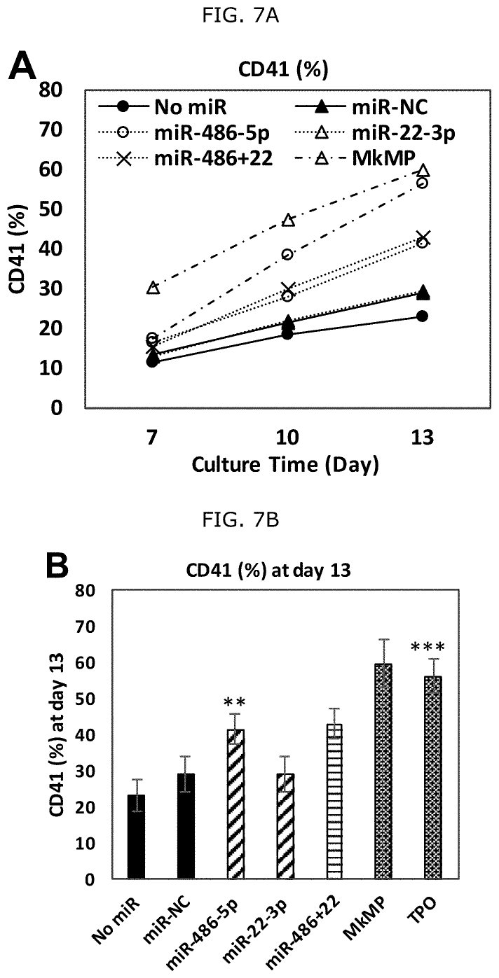

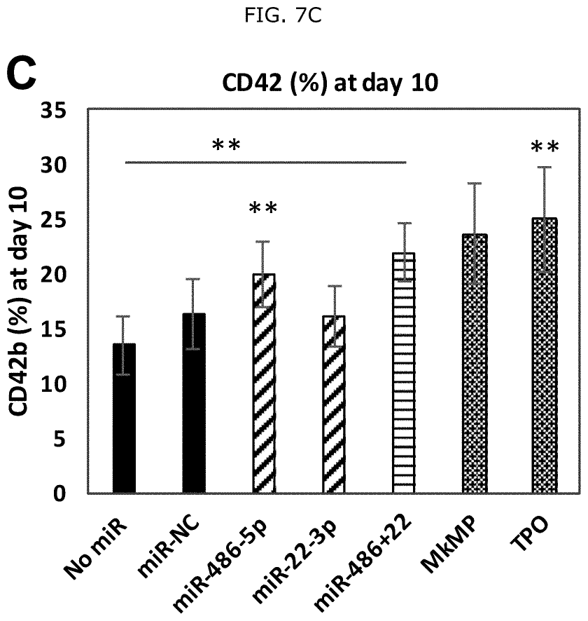

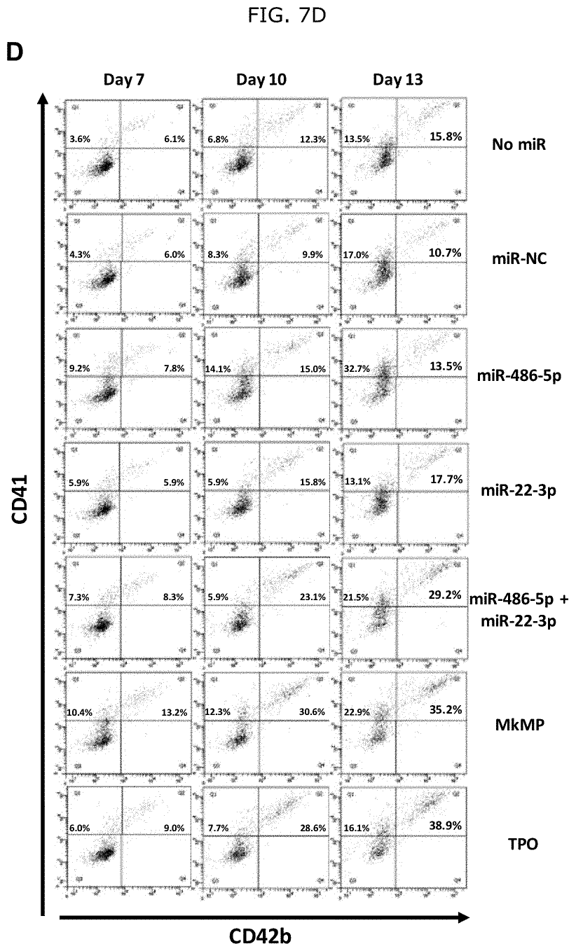

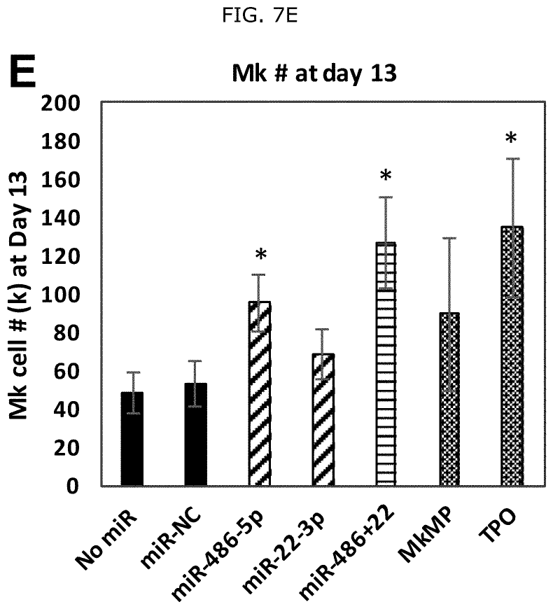

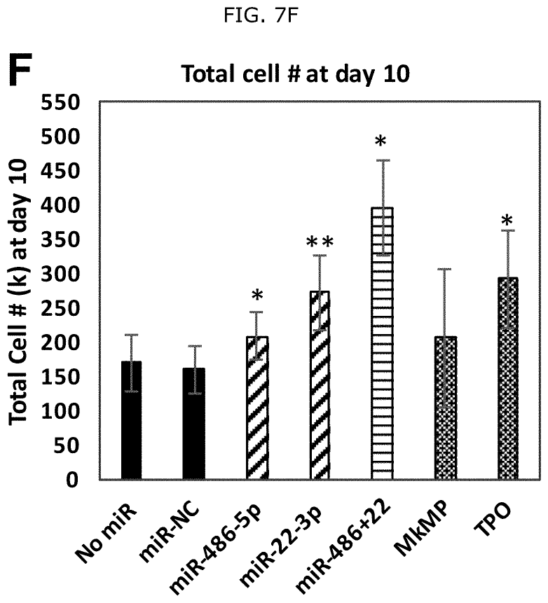

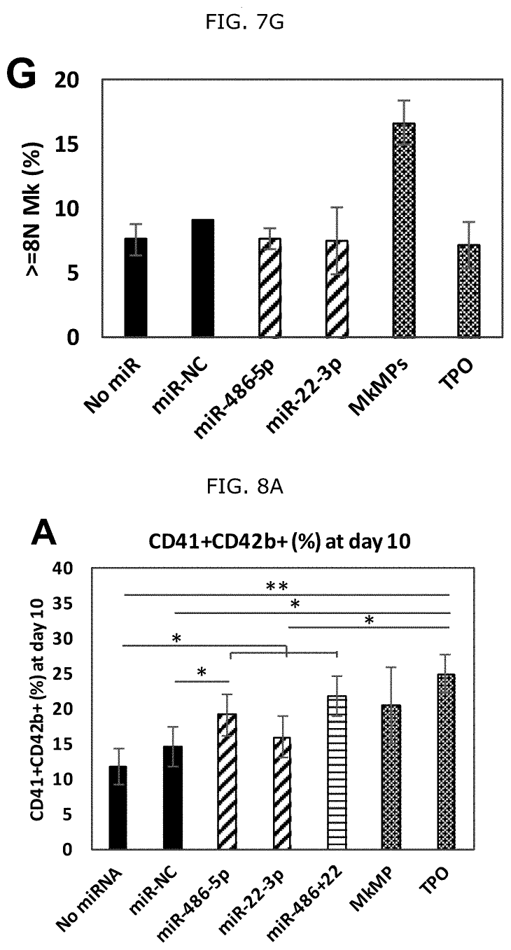

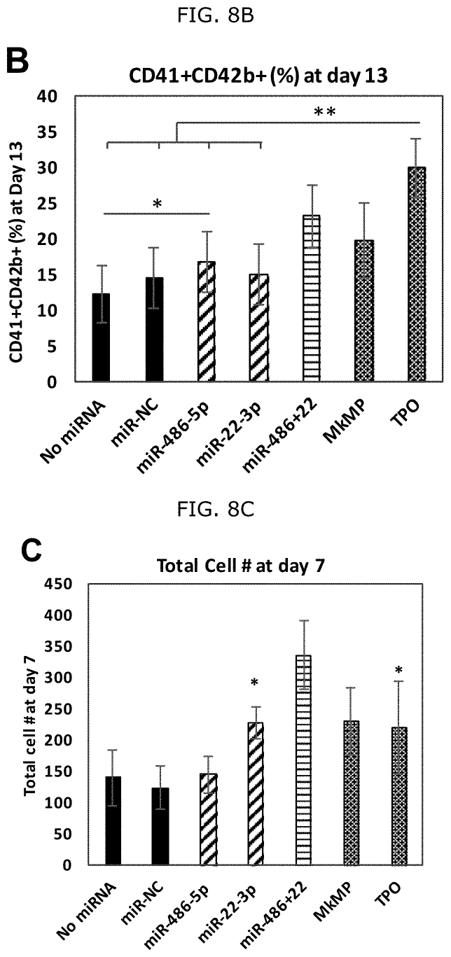

Combinations of small RNAs (siRNAs or miRs) have been shown to improve cell proliferation, and alter cellular phenotypes. We thus hypothesized that MkMP-induced Mk differentiation of CD34⁺ HSPCs might be mediated by miR-486-5p and miR-22-3p acting together. We thus examined their combinatorial targeting on CD41⁺ or CD42b⁺ expression, Mk-cell count, and total cell count in TPO-free cultures post transfection of CD34⁺ cells. Co-culture of CD34⁺ HSPCs with MkMPs, or CD34⁺ HSPC culture supplemented with TPO served as positive controls; all CD34⁺ cells were exposed to the electroporation conditions used for miR transfection, which, as would be expected, would lead to attenuated culture outcomes in terms of Mk differentiation and expansion. miR-486-5p significantly promoted Mk differentiation of CD34⁺ HSPCs with 41% of cells expressing CD41, while 43% of cells co-transfected with miR-486-5p and miR-22-3p were CD41⁺ (FIGS. 7A and 7B). Notably, compared to positive controls (MkMP, TPO), which resulted in 60% or 56% of the cells expressing CD41 by day 13, the miR-486-5p mimic alone achieved ca. 70% of their effect. Cells in each condition were also examined for expression of CD42b (a late Mk marker) at day 10. miR-486-5p or the combination of miR-486-5p and miR-22-3p mimics significantly enhanced CD42b expression with 20% and 22% of cells expressing CD42b (FIG. 7C), indicating that miR-486-5p mediates Mk maturation. Representative quadrant plots (FIG. 7D) demonstrate the combinatorial effect of miR-486-5p and miR-22-3p on CD41⁺CD42b⁺ expression (upper-right quadrant, 29.2% double positive) at day 13, which is ca. 2 and 3 times higher than the “No miR” and “miR-NC” negative controls, respectively, and closer to that of the two positive controls (MkMP, 35.2%; TPO, 38.9%)(the percent of CD41⁺CD42b⁺ cells at days 10 and day 13 are plotted in FIGS. 8A and 8B). Moreover, miR-486-5p targeting resulted in a significant increase (up to 98%) in the number of total Mks at day 13 compared to negative controls (FIG. 7E). Compared to negative controls, the combinatorial effect of miR-486-5p and miR-22-3p resulted in a ca. 2.6-fold increase of total Mk cells at day 13, virtually matching the effect of TPO (FIG. 7E). Similar to the pilot study, miR-22-3p significantly enhanced cell proliferation, with up to 71% of an increase on total cell numbers, compared to negative controls (FIGS. 7F and 8C). The combinatorial targeting of miR-486-5p and miR-22-3p resulted in a 2.5-fold increase in total cell numbers, compared to negative controls (FIG. 7F) and a 33% increase in total Mk cells compared to miR-486-5p alone (FIG. 7E). These results demonstrate that the targeting by combinations of miRs enriched in MkMPs induces megakaryocytic differentiation in the absence of TPO, a novel and unexpected finding.

6. Microscopic analysis on the impact of miR on Mk differentiation and maturation of CD34⁺ HSPCs.

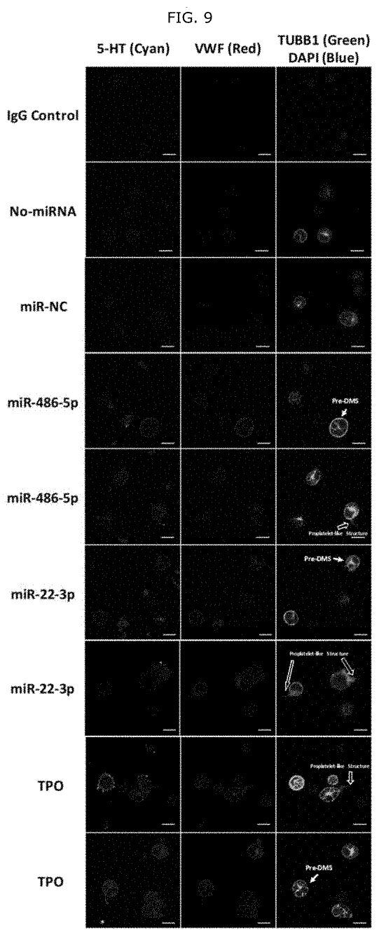

To further examine if miR-486-5p or miR-22-3p is capable of promoting late megakaryocytic differentiation of CD34⁺ HSPCs, we first examined polyploidization at day 16 for key experimental conditions (CD34⁺ transfected with miR-486-5p, miR-22-3p, or control No miR, miR-NC, as well as control CD34⁺ cells cultured post-electroporation with TPO or MkMPs) (FIG. 7G). These data show that electroporation suppresses Mk polyploidization under all conditions, and that only MkMPs can partially rescue this suppression. Polyploidization is statistically identical for all other conditions. Next, we examined the cells at day 13 by confocal microscopy for expression of beta-1 tubulin (TUBB1), von Willebrand factor (vWF), and serotonin (5-HT), which are indicators of Mk maturation and platelet formation. The images look similar for the three conditions: miR-486-5p, miR-22-3p & TPO (FIG. 9). Despite the suppressive effect of electroporation on polyploidization, both miR-486-5p-transfected and miR-22-3p-transfected cells displayed a few proplatelet-like structures (white open arrows in FIG. 9), vWF and 5-HT expression similar to that of the TPO-only culture. We also identified pre-demarcation membrane system (DMS) structures (white arrows in FIG. 9) in miR-486-5p and miR-22-3p-transfected cells, and cells from TPO culture, indicating megakaryocytic maturation. These results suggest that miR-486-5p and miR-22-3p impart megakaryocytic characteristics similar to those of TPO. Only 5-HT expression was slightly higher in the TPO-induced culture than “miR-486-5p” or “miR-22-3p” culture. The negative controls (No miR, miR-NC) displayed no 5-HT or VWF expression. Due to the suppressive effect of electroporation on polyploidization, the impact of miR-486-5p and miR-22-3p on HSPC development only into early megakaryopoiesis is inconclusive. It is possible that they may also impart megakaryocytic maturation. There is no literature that would suggest that CD41/CD42 expression does not lead to Mk maturation. Overall, these data suggest that miR-486-5p and miR-22-3p may be the key MkMP molecules through which MkMPs program CD34⁺ HSPC into Mk differentiation.

7. miR-486-5p in MkMPs serve as a key element mediating MkMP-induced megakaryocytic differentiation.

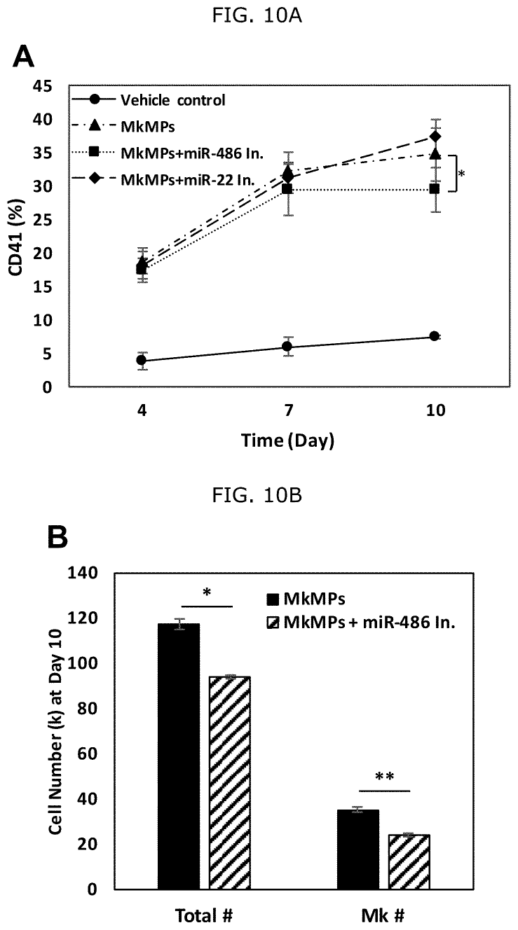

Previously, we had shown that co-culture of CD34⁺ cells with MkMPs loaded with exogenous miR-486-5p enhances megakaryocytic differentiation (22% higher fraction of CD41⁺ cells) compared to the co-culture with native MkMPs or MkMPs loaded with miR-NC. To further validate the importance of native miR-486-5p or miR-22-3p in MkMPs, here, we performed an experiment, where CD34⁺ cells were co-cultured with MkMPs loaded inhibitors of miR-486-5p or miR-22-3p (8 μM solutions for electroporation). Compared to MkMPs control, loading of miR-486-5p inhibitor to MkMPs significantly reduced the percentage of CD41⁺ cell by 14% (from 34.7% to 29.4%) at day 10, but the miR-22-3p inhibitor had no effect (FIG. 10A). Since miR-486-5p is the most abundant miR in MkMPs (FIG. 4), we hypothesized that higher levels of miR-486-5p inhibitors might be needed to achieve a stronger effect. Thus, when we doubled the concentration of miR-486-5p inhibitor for loading via electroporation MkMPs to 16 μM, it resulted in significantly lower number of total cells or Mk cells at day 10 (FIG. 10B). Overall, these results further strengthen the evidence for a role of miR-486-5p in MkMPs in programming CD34⁺ HSPCs into Mk differentiation.

8. Use of signaling-pathway inhibitors suggests that JNK and PI3K/Akt/mTOR signaling regulate MkMP-induced Mk differentiation of HSPCs.

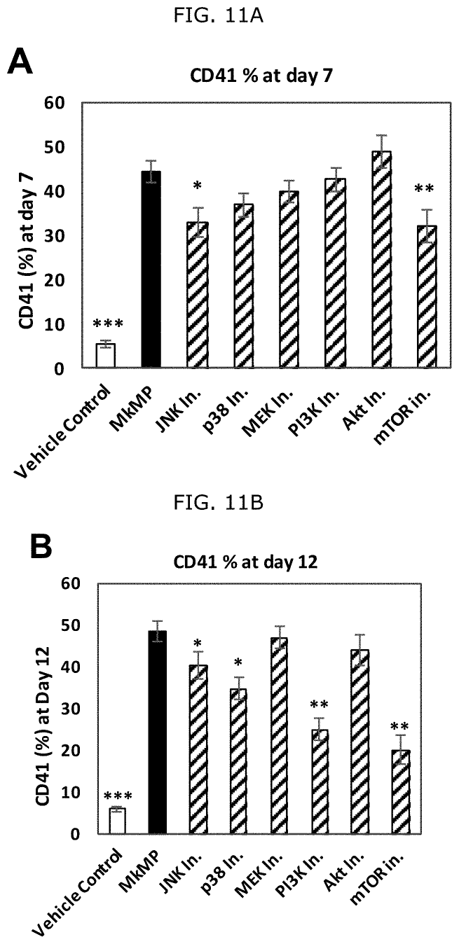

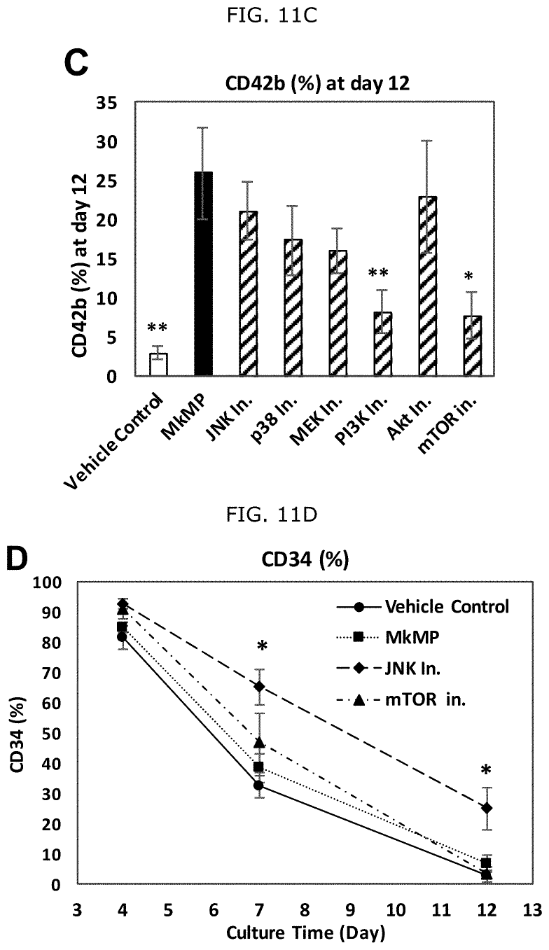

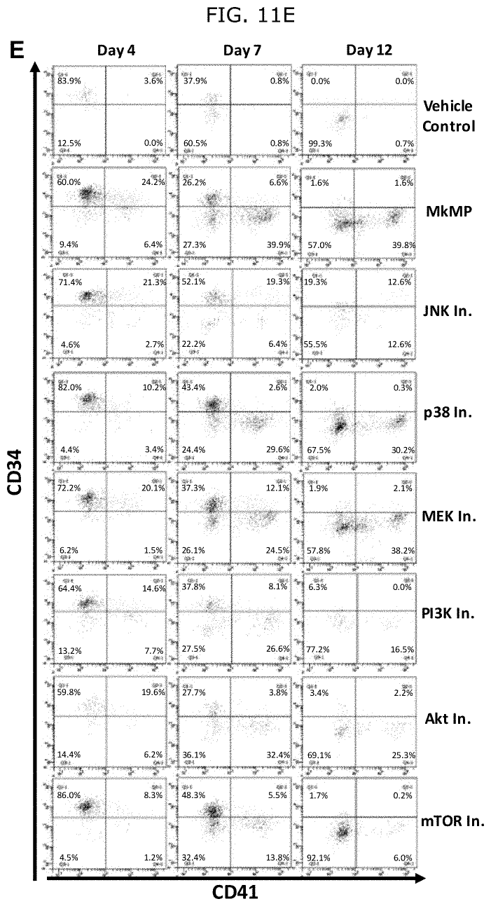

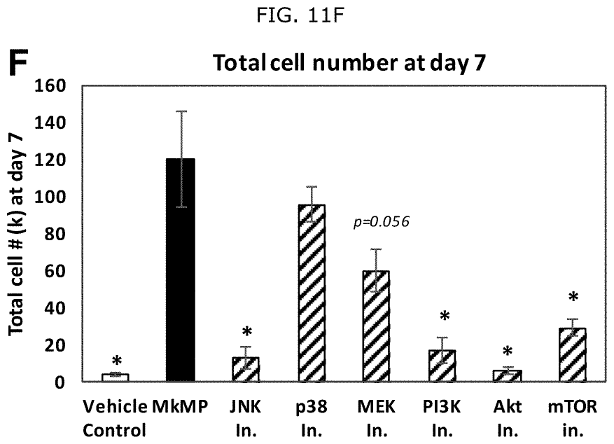

To further investigate the effects of MkMPs in promoting megakaryocytic differentiation of CD34⁺ HSPCs, and how this might relate to the miR content of MkMPs, we next probed likely signaling pathways using kinase inhibitors. We started by examining signaling pathways known to be involved in TPO signaling as summarized on the Introduction. Briefly, CD34⁺ HSPCs were pretreated with kinase inhibitors of chosen signaling pathways (JNK, p38, MEK, PI3K, Akt and mTOR inhibitors, Table 6) for 30 min before they were co-cultured with MkMPs at the ratio of 10 MkMPs/cell. Inhibitors were replenished at days 3 and 7. CD41, CD42b and CD34 expression, and cell numbers were measured at days 4, 7, and 12. These kinase inhibitors are known to affect signaling by preventing activation (e.g., phosphorylation) of downstream molecules. Therefore, we expected that if a particular signaling pathway is involved in generating the phenotypic impact of MkMPs on CD34⁺ HSPCs, then we would observe reduced-phosphorylation of downstream molecules. Compared to MkMP control, the JNK (SP600125) and mTOR (rapamycin) inhibitors significantly suppressed Mk differentiation decreasing CD41 expression at day 7 from 44.3% (MkMP) to 32.8% (JNK) and 32.1% (mTOR), respectively (FIG. 11A), while the p38 (SB203580), JNK, PI3K (LY-294002), or mTOR inhibitors significantly suppressed CD41 expression at day 12 (FIG. 11B). CD42b expression was significantly inhibited by PI3K or mTOR inhibition (FIG. 11C), suggesting that MkMPs promote Mk maturation via PI3K/mTOR signaling. JNK inhibition resulted in a higher fraction of cells expressing CD34 compared to MkMP or vehicle controls at both day 7 and day 12 (FIG. 11D).

To further assess the impact of signaling inhibitors, representative flow-cytometric quadrant plots (FIG. 11E) display the transition of MkMP-induced Mk differentiation of CD34⁺ cells, from CD34⁺CD41⁻ (upper-left) to CD34⁺CD41⁺ (upper-right), and on to CD34⁻CD41⁺ (lower-right). For example, by treating the MkMP-HSPC co-culture with JNK, p38, or mTOR inhibitors, at day 7, a larger fraction of cells was CD34⁺CD41⁻ (52.1%, 43.4%, and 48.3%, respectively) compared to MkMP control (26.2%), thus indicating that JNK, p38, or mTOR signaling is involved in the early MkMP-induced differentiation of CD34⁺ HSPCs. At day 12, 39.8% of cells were CD34⁻CD41⁺ in the MkMP control, while only 12.6%, 16.5%, 25.3%, or 6.0% of the cells were CD34⁻CD41⁺ using JNK, PI3K, Akt, or mTOR inhibitors. Treatment with the JNK, PI3K, Akt, or mTOR inhibitors significantly inhibited cell growth and decreased total-cell numbers by more than 75% at day 7 of co-culture (FIG. 11F). Taken together, these data suggest that JNK, p38, PI3K, Akt and mTOR signaling are involved in MkMP-induced Mk differentiation of CD34⁺ HSPCs. PI3K and mTOR signaling appears to be involved in MkMP-promoted Mk maturation (FIG. 11C), while JNK, PI3K, Akt, or mTOR signaling play an important role in MkMP-mediated cell proliferation (FIG. 11G).

9. MkMPs target JNK-mediated PI3K/Akt/mTOR signaling in HSPCs.

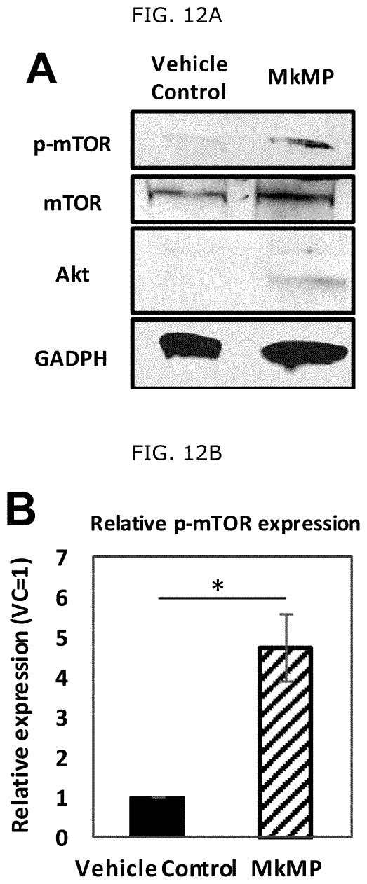

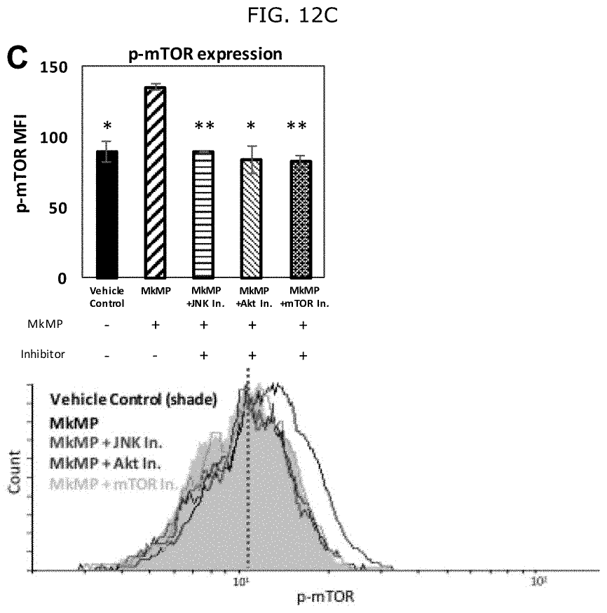

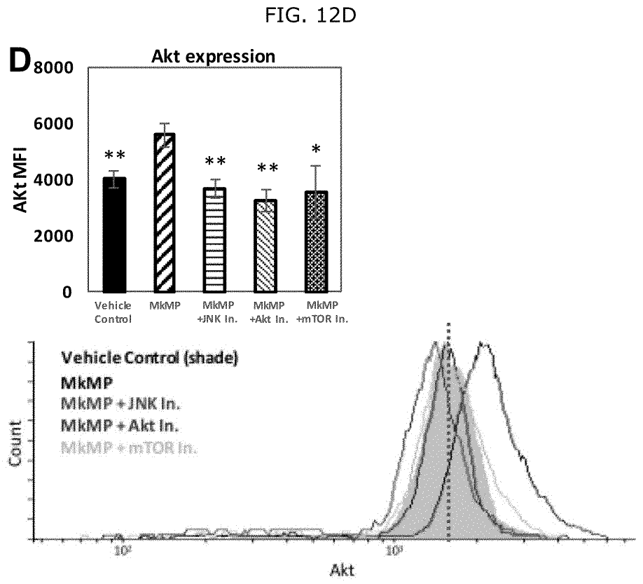

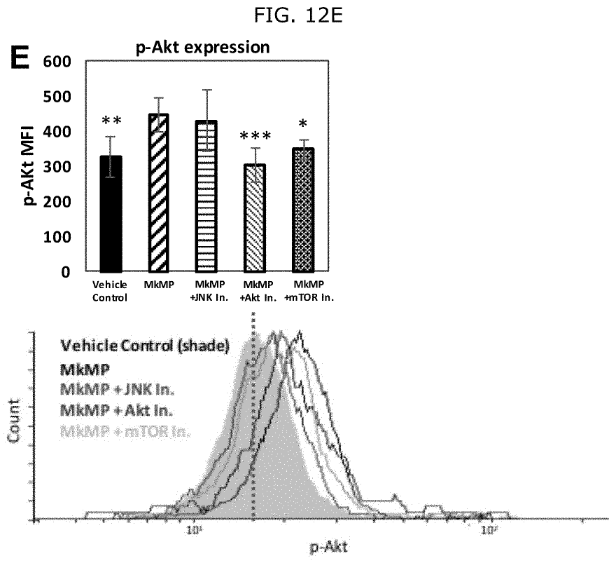

Based on our findings above, both JNK and PI3K/Akt/mTOR appear to be involved in MkMP-induced cell proliferation and Mk differentiation (FIG. 11). While PI3K/Akt signaling has been shown to be involved in TPO-mediated Mk differentiation, very little has been reported regarding JNK signaling in megakaryopoiesis. To pursue these findings further, we first examined expression of total and phosphorylated Akt and mTOR by immunoblotting. We expected lower phosphorylation levels of Akt or mTOR when using the Akt (Wortmannin) or mTOR inhibitor (Rapamycin), respectively. CD34⁺ HSPCs were pre-incubated with or without JNK, Akt, or mTOR inhibitors (Table 6), and co-cultured with MkMPs for 24 hours. The data (FIG. 12A) show that total Akt expression and phosphorylated mTOR (p-mTOR) were higher (p-mTOR by 4.7 fold (FIG. 12B)) in HSPCs co-cultured with MkMPs, but there were no changes on total mTOR levels. With the limited number of cells in CD34⁺ HSPCs cultures using signaling inhibitors, quantitating the very low expression levels of phosphorylated Akt became a significant challenge (data no shown). We thus examined total and phosphorylated Akt and mTOR levels using flow cytometry, which requires relatively few cells (FIGS. 6C-E). p-mTOR and Akt expression were significantly higher in the MkMP co-cultures, while treatment with JNK, Akt or mTOR inhibitors brought the expression levels back to that of vehicle control (FIGS. 12C and 12D). Total mTOR remained unaffected under all conditions (data not shown). Higher increase of p-mTOR levels was detected from immunoblotting (FIG. 12B) than by flow-cytometric analysis (FIG. 12C) likely because immunoblotting examines protein levels in both live and dead cells, while only live cells are examined by flow cytometry. Flow-cytometric analysis (FIG. 12E) suggests that MkMPs also activate Akt phosphorylation. However, the level of p-Akt was not affected by the JNK inhibitor. These results suggest that, in CD34⁺ HSPCs stimulated by MkMPs, total Akt expression, but not total mTOR expression, is impacted by JNK signaling.

10. PTEN mediated MkMP-induced signaling in CD34⁺ HSPCs.

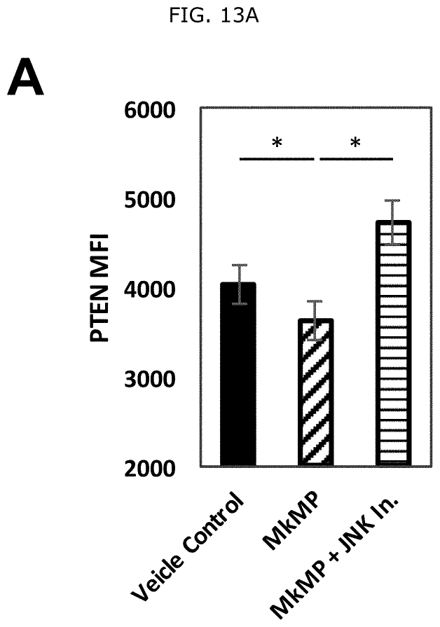

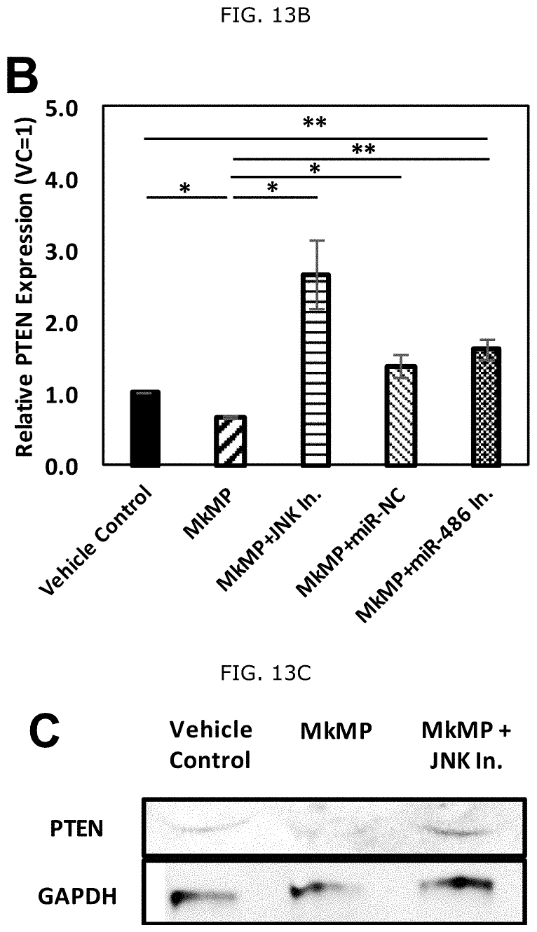

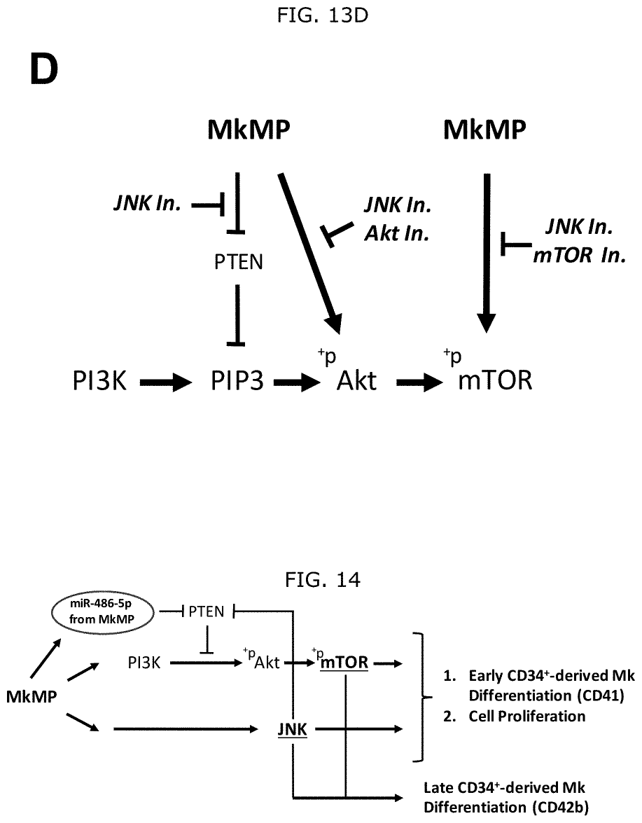

Phosphatase and TENsin homolog (PTEN) is a negative regulator of PI3K/Akt signaling in HSC development. Loss of PTEN results in enhanced cellular proliferation and megakaryopoiesis due to overactive of PI3K/Akt signaling. We hypothesized that activation of PI3K/Akt/mTOR signaling by MkMPs might be PTEN mediated. We thus examined the mRNA level of PTEN by quantitative PCR, and PTEN protein level by flow cytometry and immunoblotting. The impact on PTEN mRNA levels appears higher than that on protein levels. Compared to the vehicle control, PTEN protein or mRNA levels were around 10% or 30% lower, respectively, in HSPCs co-cultured with MkMPs (FIGS. 13A and 13B). This effect was strengthened by the finding that the JNK inhibitor restored PTEN protein or mRNA levels by 17% or 260% above vehicle-control levels (FIGS. 13A-C), respectively. These statistically-significant but relatively low-impact effects on PTEN protein expression would be expected given that the measurements were done within the first 24 hours of the co-culture, rather than late in the co-culture. Importantly, co-culture with MkMPs loaded with miR-486-5p inhibitor resulted in 2.4 or 1.2 fold higher PTEN-mRNA levels, compared to native MkMPs or MkMPs loaded with miR-NC, respectively (FIG. 13B), thus suggesting that there is a relationship between miR-486-5p in MkMPs and PTEN in HSPCs. To sum, our data suggest that MkMPs regulate Akt/mTOR signaling by enhancing Akt expression and activating Akt/mTOR, possibly mediated via JNK signaling including by PTEN targeting (FIG. 13D).

### DISCUSSION

Synergistic action of two miRs mimicking TPO signaling. EV miRs are important mediators of EV-based cell-to-cell communication. Such miRs are either highly abundant or/and highly enriched in EVs. Here, we used RNAseq to identify miRs highly enriched in MkMPs, and examined the role of the most abundant miRs in promoting HSPC differentiation and cell proliferation (FIG. 4). As discussed, only few studies have examined combinatorial effects of two or more miRs on cell fate. Our results suggest that miR-486-5p induces Mk commitment and early development (CD41⁺ cells) of HSPCs (FIGS. 7A and 7E), miR-22-3p promotes cell proliferation (FIGS. 7F and 8C) and Mk maturation (FIG. 7D), and, combinatorically, Mk maturation (FIGS. 6C and 6E), and Mk and total-cell expansion (FIGS. 6E & 6F). CD34⁺ transfected with miR-486-5p or miR-22-3p displayed Mk characteristics, including vWF/5-HT expression, DMS and proplatelet structures (FIG. 9), similar to TPO-induced Mks. Together, miR-22-3p and miR-486-5p appear to mimic the TPO-induced Mk differentiation of CD34⁺ HSPCs with the notable outcome of Mk numbers being comparable to those in TPO-induced megakaryopoiesis (FIG. 7E). This is the first report of two miRs inducing and co-regulating megakaryopoiesis of CD34⁺ HSPCs. However, other abundant miRs (FIG. 6), less abundant miRs, or other small RNAs (piRNA, snoRNA, non-snoRNA, Tables 1-3) would synergize with these two miRs to further promote Mk differentiation mimicking in full or even exceeding the effect of TPO.

Several miRs have been previously reported as regulators of megakaryopoiesis. These and all in vitro studies of miRs in megakaryopoiesis were carried out in the presence of TPO. To our knowledge, there have been no reports of miRs promoting megakaryocytic differentiation of CD34⁺ HSPCs in the absence of TPO.

miR-486-5p has recently been shown to regulate erythroid differentiation and survival of cord blood CD34⁺ cells via Akt signaling, both in vitro and in vivo. Conflicting roles for miR-22 have been reported in the development of hematopoietic malignancies, as a tumor suppressor or oncogenic. Most recently, Weiss and Ito reported that miR-22 is upregulated during in vivo murine megakaryopoiesis, and that miR-22 knockout impairs megakaryocytic differentiation, while miR-22 overexpression promotes megakaryocytic differentiation in the K562 cell line. Their results suggest a similar miR-22 role as we report here (FIG. 7D). miR-22-3p was also most recently shown to regulate mTOR signaling by targeting eukaryotic translation initiation factor 4E-binding proteins (eIF4EBP3) in human cervical squamous carcinoma cells.

While miRs have been previously identified in human platelets, it is difficult to compare our RNAseq data (Table 5) to other studies, due to the fact that the data are collected and/or analyzed differently. For example, from microarray screening, miR-126, miR-197, miR-223, miR-24, and miR-21 were found to be the most highly expressed miRs in platelets, which is different from our ranking of top miRs in platelets (Table 5). Nagalla et al. have also published a miR profile of human platelets from microarray analysis. Depending on the tool used for miR analysis, the ranking of miRs in platelets varies significantly. The ranking of miRs in platelets from our data largely correlates with the data from the study of Kaudewitz & Skroblin, which used a similar strategy based on RNAseq. Lastly, Juzenas et al. provide a comprehensive miR dataset for leukocytes and erythrocytes.

JNK and Akt/mTOR signaling in MkMP-induced Mk differentiation of CD34⁺ HSPCs & a proposed model linking miR targeting to megakaryopoietic signaling. Multiple signaling pathways are engaged in TPO-induced megakaryopoiesis, including those of PI3K/Akt, MAPK, and Jak/STAT. From the kinase inhibitor studies (FIGS. 11-13), we identified the role of JNK and PI3K/Akt/mTOR signaling. Although it has been shown that JNK can be activated by TPO, there is no known role of JNK signaling in Mk development. Using a JNK inhibitor in the co-culture of MkMPs with CD34⁺ HSPCs, CD34 expression was significantly maintained (FIG. 11D) and Mk numbers at day 7 were significantly reduced, thus indicating that JNK signaling mediates early Mk differentiation and expansion in this MkMP-induced phenotype. Maintenance of CD34 expression is consistent with the recent finding that treatment of human cord blood CD34⁺ cells with JNK inhibitors (JNK-IN-8 or SP600125) enhanced the self-renewal of HSCs. We have also demonstrated that JNK signaling is involved in shear-induced Mk maturation and platelet production.

mTOR is a major regulator of Mk development and maturation. Here, we showed that CD41 expression was significantly lower at days 7 and 12 in cells treated with an mTOR inhibitor prior to co-culture with MkMPs (FIGS. 11A & 11B). Akt expression was upregulated, and Akt and mTOR were phosphorylated in HSPCs co-cultured with MkMPs (FIGS. 12C-E), possibly through PTEN targeting. These results suggest that MkMPs activate Akt/mTOR signaling in HSPCs to induce Mk differentiation. Surprisingly, downregulation of PTEN expression by MkMPs was abolished upon treatment with a JNK inhibitor (FIGS. 13A-C), together with reduced total Akt expression (FIG. 12D) and mTOR phosphorylation (FIG. 12C). These findings suggest that there is crosstalk between JNK and PI3K/Akt/mTOR signaling. A similar case has been previously reported, namely the negative regulation of PTEN by c-Jun, a downstream molecule in JNK signaling. To sum, our data suggest that it is possible that MkMP-induced Mk differentiation of CD34⁺ HSPCs is regulated by the circuit of JNK and Akt/mTOR signaling.

miR-486-5p have been shown to target PTEN and PI3K/Akt signaling in several cell types. Specifically, miR-486-5p regulates Akt signaling, cell proliferation and survival in cord-blood derived CD34⁺ cells by directly targeting PTEN. It is possible that miR-486-5p from MkMPs directly targets PTEN and activates PI3K/Akt/mTOR signaling in CD34⁺ HSPCs. Our data suggest that miR-22-3p plays an important role in cell proliferation and Mk maturation (FIG. 14). The former is consistent with the finding that conditional miR-22 expression in the murine hematopoietic compartment increases hematopoietic stem cell self-renewal by directly targeting the tumor suppressor TET2. The latter is consistent with the finding that miR-22 promotes megakaryopoiesis by repressing the repressive transcription factor GFI1. The proposed model of FIG. 7 captures and integrates our data and the current knowledge.

TABLE 1
Top 5 expressed human piRNA in MkMPs and PLPs

| | MkMPs | | |
|---|---|---|---|
| | NCBI | | |
| Rank | Accession | piRNA ID | Fraction |
| 1 | DQ571224.1 | hsa_piR_001312 | 19.4 |
| 2 | DQ970956.1 | hsa_piR-000765 | 18.4 |
| 3 | DQ597916.1 | hsa_piR-020326 | 18.4 |
| 4 | DQ592931.1 | hsa_piR_016658 | 10.2 |
| 5 | DQ594465.1 | hsa_piR_017724 | 9.1 |
| | PLPs | | |
| | NCBI | | |
| Rank | Accession | piRNA ID | Fraction |
| 1 | DQ571224.1 | Hsa_piR_001312 | 22.9 |
| 2 | DQ970956.1 | hsa_piR-000765 | 21.7 |
| 3 | DQ597916.1 | hsa_piR-020326 | 21.7 |
| 4 | DQ594465.1 | hsa_piR_017724 | 8.8 |
| 5 | DQ575882.1 | hsa_piR_004308 | 3.8 |

Distinct from miRs, Piwi-interacting RNAs (piRNAs) are 24-31 nt in length. As described in the Materials and Methods, we map cDNA sequences (18-40 nt) to human piRNA sequences from the piRNABank (http://pirnabank.ibab.ac.in/). Sequencing data analysis shows that 149 and 152 of a total 458 piRNAs in piRNABank were expressed (average CPM >=1) in MkMPs and PLPs, respectively. The top 5 expressed piRNAs comprised 76% and 79% of total piRNAs carried by MkMPs and PLPs, respectively. NCBI accession for each piRNA is referred in the table.

TABLE 2
Top 10 expressed human snoRNA (with NCBI Gene ID) in MkMPs
and PLPs.

| | MkMP | | | PLP | | |
|---|---|---|---|---|---|---|
| | NCBI | | | NCBI | | |
| | Gene | | Avg | Gene | | Avg |
| Rank | ID | snoRNA ID | CPM | ID | snoRNA ID | CPM |
| 1 | 9297 | SNORD29 | 147576 | 9297 | SNORD29 | 282862 |
| 2 | 606500 | SNORD68 | 125014 | 692227 | SNORD104 | 178695 |
| 3 | 692227 | SNORD104 | 124403 | 606500 | SNORD68 | 97625 |
| 4 | 26809 | SNORD42A | 46614 | 26809 | SNORD42A | 50157 |
| 5 | 9302 | SNORD26 | 27796 | 26806 | SNORD44 | 28201 |
| 6 | 692212 | SNORD99 | 23774 | 692212 | SNORD99 | 23767 |
| 7 | 26806 | SNORD44 | 21651 | 9302 | SNORD26 | 22674 |
| 8 | 26799 | SNORD50A | 17159 | 26807 | SNORD43 | 21394 |
| 9 | 26807 | SNORD43 | 16061 | 619567 | SNORD2 | 17408 |
| 10 | 619567 | SNORD2 | 14935 | 26799 | SNORD50A | 16937 |

Based on the expression level, among small RNAs of 40-150 nt length, more than 99% of the mapped small RNAs were small nucleolar RNA (snoRNAs)

TABLE 3
Top 10 expressed human non-snoRNA (with NCBI Gene ID) in
MkMPs and PLPs

| | | MkMP | | | PLP | |
|---|---|---|---|---|---|---|
| | NCBI | non-snoRNA | Avg | NCBI | non-snoRNA | Avg |
| Rank | Gene ID | ID | CPM | Gene ID | ID | CPM |
| 1 | 100151683 | RNU4ATAC | 1205 | 267010 | RNU12 | 933 |
| 2 | 6090 | RNY5 | 656 | 109623460 | MIR3607 | 632 |
| 3 | 56664 | VTRNAl-1 | 600 | 100151683 | RNU4ATAC | 284 |
| 4 | 406955 | MIR181B1 | 443 | 407010 | MIR23A | 265 |
| 5 | 267010 | RNU12 | 411 | 56664 | VTRNA1-1 | 79 |
| 6 | 100126299 | VTRNA2-1 | 249 | 6090 | RNY5 | 51 |
| 7 | 109623460 | MIR3607 | 235 | 406948 | MIR15A | 32 |
| 8 | 100302143 | MIR1248 | 147 | 677771 | SCARNA4 | 29 |
| 9 | 677771 | SCARNA4 | 133 | 677780 | SCARNA11 | 26 |
| 10 | 407010 | MIR23A | 80 | 26824 | RNU11 | 25 |

TABLE 4
Significantly enriched (p < 0.01; differential expression ≥
two-fold) miRs (CPM > 100) in MkMPs compared to Mks.
NCBI Gene ID of each miR included

| NCBI | | MkMPs | Mks | Fold | |
|---|---|---|---|---|---|
| Gene ID | miRNA | (CPM) | (CPM) | Change | FDR |
| 406980/ | mir-19b-1//mir- | 1999.33 | 378.86 | 2.43 | 0.00025 |
| 406981 | 19b-2_3P | | | | |
| 406955/ | mir-181b-1//mir- | 1869.99 | 363.50 | 2.37 | 0.00051 |
| 406956 | 181b-2 | | | | |
| 494327 | mir-378a_3P | 1766.13 | 281.71 | 2.92 | 0.00009 |
| 406979 | mir-19a_3P | 986.87 | 170.89 | 2.66 | 0.00012 |
| 407030 | mir-30b | 873.83 | 129.73 | 2.99 | 0.00000 |
| 406952 | mir-17 | 452.59 | 101.47 | 2.01 | 0.00172 |
| 442904 | mir-335 | 394.39 | 44.40 | 3.93 | 0.00000 |
| 406900 | mir-106b | 388.20 | 70.16 | 2.47 | 0.00001 |
| 442910 | mir-345 | 361.60 | 66.55 | 2.49 | 0.00081 |
| 406947 | mir-155 | 343.28 | 54.40 | 2.91 | 0.00003 |
| 406957 | mir-181c | 324.84 | 64.18 | 2.29 | 0.00005 |
| 406982 | mir-20a | 288.40 | 60.84 | 2.15 | 0.00059 |
| 100302117/ | mir-320b-1//mir- | 266.37 | 47.07 | 2.57 | 0.00027 |
| 100313769 | 320b-2_3P | | | | |
| 574411 | mir-451a | 190.90 | 27.36 | 3.18 | 0.00000 |
| 100616247 | mir-151b_3P | 152.06 | 23.24 | 2.89 | 0.00006 |
| 574452 | mir-494_3P | 135.02 | 28.49 | 2.13 | 0.00177 |
| 442914 | mir-369_3P | 121.11 | 23.72 | 2.23 | 0.00539 |
| 442913 | mir-376c_3P | 118.30 | 25.94 | 2.03 | 0.00204 |

TABLE 5
Top 10 most abundant miRNAs in MkMPs, Mk cells, PLTs, or PLPs.

| | | miRNA in | | | | miRNA | | |
|---|---|---|---|---|---|---|---|---|
| Rank | NCBI Gene ID | Mks | CPM | % | NCBI Gene ID | in MkMPs | CPM | % |
| 1 | 406966 | miR-191- | 231441 | 23.1 | 723876 | miR- | 164087 | 16.4 |
| | | 5p | | | | 486-5p | | |
| 2 | 723876 | miR-486- | 195803 | 19.6 | 406966 | miR- | 117782 | 11.8 |
| | | 5p | | | | 191-5p | | |
| 3 | 407056 | miR-99b- | 164315 | 16.4 | 407015/407016 | miR- | 91774 | 9.2 |
| | | 5p | | | | 26a-5p | | |
| 4 | 574447 | miR- | 65671 | 6.6 | 406888/406889 | let-7f-5p | 68251 | 6.8 |
| | | 146b-5p | | | | | | |
| 5 | 407015/407016 | miR-26a- | 39625 | 4.0 | 407048/407049 | miR- | 44480 | 4.4 |
| | | 5p | | | | 92a-3p | | |
| 6 | 406888/406889 | let-7f-5p | 23623 | 2.4 | 406913 | miR- | 44218 | 4.4 |
| | | | | | | 126-5p | | |
| 7 | 406913 | miR-126- | 22642 | 2.3 | 407004 | miR-22- | 43152 | 4.3 |
| | | 5p | | | | 3p | | |
| 8 | 406938 | miR- | 21993 | 2.2 | 406991 | miR-21- | 38617 | 3.9 |
| | | 146a-5p | | | | 5p | | |
| 9 | 406991 | miR-21- | 20197 | 2.0 | 574447 | miR- | 34455 | 3.4 |
| | | 5p | | | | 146b-5p | | |
| 10 | 406940 | miR- | 17054 | 1.7 | 406995/406996 | miR- | 33752 | 3.4 |
| | | 148a-3p | | | | 181a-5p | | |
| | | Other | | 19.8 | | Other | | 31.9 |
| | | miRNAs | | | | miRNAs | | |
| | | miRNA in | | | | miRNA in | | |
| Rank | NCBI Gene ID | PLTs | CPM | % | NCBI Gene ID | PLPs | CPM | % |
| 1 | 406966 | miR-191- | 263871 | 26.4 | 406966 | miR- | 162495 | 16.2 |
| | | 5p | | | | 191-5p | | |
| 2 | 723876 | miR-486- | 80546 | 8.1 | 723876 | miR- | 146874 | 14.7 |
| | | 5p | | | | 486-5p | | |
| 3 | 406888/406889 | let-7f-5p | 76283 | 7.6 | 407015/407016 | miR- | 84857 | 8.5 |
| | | | | | | 26a-5p | | |
| 4 | 407056 | miR-99b- | 71050 | 7.1 | 406888/406889 | let-7f-5p | 64288 | 6.4 |
| | | 5p | | | | | | |
| 5 | 406902 | miR-10a- | 54139 | 5.4 | 574447 | miR- | 49986 | 5.0 |
| | | 5p | | | | 146b-5p | | |
| 6 | 407015/407016 | miR-26a- | 51712 | 5.2 | 406913 | miR- | 43506 | 4.4 |
| | | 5p | | | | 126-5p | | |
| 7 | 406938 | miR- | 43647 | 4.4 | 407056 | miR- | 42826 | 4.3 |
| | | 146a-5p | | | | 99b-5p | | |
| 8 | 407048/407049 | miR-92a- | 43032 | 4.3 | 406991 | miR-21- | 40419 | 4.0 |
| | | 3p | | | | 5p | | |
| 9 | 574447 | miR- | 25104 | 2.5 | 407004 | miR-22- | 32727 | 3.3 |
| | | 146b-5p | | | | 3p | | |
| 10 | 406995/406996 | miR- | 23932 | 2.4 | 406995/406996 | miR- | 25520 | 2.6 |
| | | 181a-5p | | | | 181a-5p | | |
| | | Other | | 26.7 | | Other | | 30.7 |
| | | miRNAs | | | | miRNAs | | |

NCBI Gene ID of each miR included.

TABLE 6
List of signaling inhibitors, their targets, and concentrations used

| Signaling | | | |
|---|---|---|---|
| Inhibitor | Target | Pathway | Concentration |
| SP600125 | JNK | MAPK | 10 uM |
| SB203580 | p38 | | 10 uM |
| PD98059 | MEK | | 10 uM |
| LY-294002 | PI3K | PI3K/Akt/mTOR | 10 uM |
| Wortmannin | Akt | | 10 uM |
| Rapamycin | mTOR | | 10 uM |

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1. A method for inducing megakaryocytic differentiation of hematopoietic stem/progenitor cells (HSPCs) without megakaryocytic microparticles (MkMPs), comprising transferring into the HSPCs an effective amount of one or more small RNAs, whereby the HSPCs differentiate into megakaryocytes.

2. The method of claim 1, wherein the HSPCs are CD34⁺.

3. The method of claim 1, wherein the HSPCs differentiate into megakaryocytes in vitro in the absence of thrombopoietin (TPO).

4. The method of claim 1, wherein the HSPCs are in a subject and differentiate into megakaryocytes in the subject.

5. The method of claim 1, wherein the one or more small RNAs comprise one or more microRNAs (miRs) selected from the group consisting of miR-486, miR-22, miR-191, miR-181, miR-378, miR-26, let-7, miR-92, miR-126, miR-92, miR-21, miR-146, miR-181, and combinations thereof.

6. The method of claim 1, wherein the one or more small RNAs comprise miR-486 and miR-22.

7. The method of claim 5, wherein the one or more small RNAs further comprise hsa_piR-001312, hsa_piR-000765, hsa_piR-020326, hsa_piR-016658, hsa_piR-017724 or a combination thereof.

8. The method of claim 5, wherein the one or more small RNAs further comprise SNORD29, SNORD68, SNORD104, SNORD42A, SNORD26, SNORD99, SNORD44, SNORD50A, SNORD43, SNORD2 or a combination thereof.

9. The method of claim 1, wherein the one or more small RNAs are synthetic.

10. The method of claim 1, wherein the one or more small RNAs are isolated from cells.

11. The method of claim 1, further comprising transferring the one or more small RNAs into the HSPCs via cellular particles.

12. The method of claim 11, wherein the cellular particles are generated by a method comprising:
(a) culturing cells in a culture medium, wherein the cells are selected from the group consisting of megakaryocytes, immature megakaryocyte cells and a combination thereof;
(b) exposing the cultured cells of step (a) to a mechanical stress to generate cellular particles,
(c) isolating the cellular particles of step (b) from the cultured cells, and
(d) loading the isolated cellular particles of step (c) with the one or more small RNAs, whereby the cellular particles carrying the one or more small RNAs are obtained.

13. The method of claim 1, further comprising transferring the one or more small RNAs into the HSPCs by transfection, electroporation, lipofection or nucleofection.

14. The method of claim 1, further comprising administering to the HSPCs a regulator of PI3K signaling pathway.

15. The method of claim 14, wherein the regulator of the PI3K signaling pathway is a positive regulator, whereby the megakaryocytic differentiation of the HSPCs is enhanced.

16. The method of claim 1, further comprising administering to the HSPCs a regulator of Akt signaling pathway.

17. The method of claim 16, wherein the regulator of the Akt signaling pathway is a positive regulator, whereby the megakaryocytic differentiation of the HSPCs is enhanced.

18. A method for enhancing megakaryocytic differentiation of hematopoietic stem/progenitor cells (HSPCs), comprising culturing HSPCs with MkMPs in the presence of an effective amount of one or more exogenous small RNAs, whereby more of the HSPCs differentiate into megakaryocytes than those cultured with the MkMPs in the absence of the one or more exogenous small RNAs.

19. The method of claim 18, wherein the one or more exogenous small RNAs comprise miR-486.

20. The method of claim 18, further comprising loading the MkMPs with the one or more exogenous small RNAs.

21. The method of claim 18, further comprising transferring the one or more exogenous small RNAs into the HSPCs by transfection, electroporation, lipofection or nucleofection.
